(19) United States
(12) Patent Application Publication
(10) Pub. No.: US 2011/0059074 A1
(43) Pub. Date: Mar. 10, 2011

(54) Knowledge-Based Proliferation Signatures and Methods of Use

(76) Inventors: Maud H.W. Starmans, Heerlen (NL); Balaji Krishnapuram, King of Prussia, PA (US); Ranand G. Seigneuric, Crimolois (FR); Harald Steck, Phoenixville, PA (US); Dimitry S.A. Nuyten, Branford, CT (US); Francesca Buffa, London (GB); Adrian Lewellyn Harris, Oxford (GB); Bradly G. Wouters, Toronto (CA); Philippe Lambin, Genappe-Bousval (BE); R. Bharat Rao, Berwyn, PA (US); Sriram Krishnan, Exton, PA (US)

(21) Appl. No.: 12/597,610

(22) PCT Filed: May 2, 2008
(86) PCT No.: PCT/US08/05705

§ 371 Date: Jun. 3, 2010

Related U.S. Application Data
(63) Continuation-in-part of application No. 12/113,481, filed on May 1, 2008, now abandoned.
(60) Provisional application No. 60/915,518, filed on May 2, 2007.

(51) Int. Cl. A61K 39/395; G01N 33/48; C12Q 1/68; C40B 30/04; C40B 40/06; A61K 31/337; A61K 31/506; A61K 31/4045; A61K 33/24; A61K 31/7048; A61K 31/475; A61K 31/519; A61K 31/704; A61K 31/675; A61K 31/513; A61K 31/437; A61K 38/16; A61P 35/00

(52) U.S. Cl. 424/133.1; 436/94; 435/6; 506/9; 506/16; 514/449; 514/252.18; 514/414; 424/649; 514/27; 514/285; 514/262.1; 514/34; 514/90; 514/274; 514/19.3; 424/145.1

(57) ABSTRACT

The present invention provides methods and compositions for predicting patient responses to cancer treatment using a proliferation gene signature. These methods can comprise measuring in a biological sample from a patient the levels of gene expression of a group of the genes designated herein. The present invention also provides for microarrays that can detect expression from a group of genes.

### CROSS-REFERENCE TO RELATED APPLICATION

This application is the U.S. national phase application of PCT International Application No. PCT/US2008/005705, filed May 2, 2008, which claims priority to U.S. Provisional Patent Application Ser. No. 60/915,518, filed May 2, 2007, and U.S. patent application Ser. No. 12/113,481, filed May 1, 2008, the contents of such applications being incorporated by reference herein.

### BACKGROUND

The ability to predict outcome and to identify key-players in biological mechanisms that lead to poor outcome, are two important objectives in cancer research. A great deal of research has been performed by means of gene expression profiling to identify gene sets (gene signatures) that can improve diagnosis and risk stratification (1). A drawback of most of the studies performed is that supervised analysis methods are utilized to acquire such signatures. Patient microarray and clinical data are directly used to find the genes that correlate with tumor type or survival. This results in gene sets with a very high prognostic value in the studied datasets. However, application in other patient datasets is limited and the overlap in selected genes of different comparable studies is small (2). If such a signature can be applied to other datasets it will be restricted to a certain patient population and cancer type. Besides, the gene sets obtained with this method are often difficult to interpret with respect to the underlying biological mechanism (3, 4). Further Dupuy et al. (5) showed in a recent review that many of these studies show flaws in methodology.

A few studies have started from another standpoint. Instead of focusing on a certain patient group, a biological process or specific environmental condition known to influence treatment response or patient outcome is taken as base. In vitro gene expression profiling is then used to identify gene sets that play an important role in these processes. This approach has a broader application because the gene sets can be used in almost every patient group. First, it can be used to investigate whether a certain process is important in a distinct cancer type or patient group. Second, it can be applied to select patients in those groups that would benefit from therapies directed to the biological process of interest (1). Examples of gene sets attained with this approach are the wound (6), hypoxia (7, 8) and “invasiveness” (IGS) (9) signatures. These studies show that the deduced signatures can be used for risk stratification in very different types of cancers (6, 7, 9, 10), presumably because of common core pathways. Recently Fan et al. (11) compared the performance of several supervised and unsupervised derived gene sets (12). Both types of signatures showed high concordance in prognostic power. Another benefit of unsupervised research is that it renders the option to identify the functional regulators in a signature that drive the studied process (13) and might reveal new targeting candidates. One of the processes studied with this method is proliferation. The rate of tumor cell proliferation is a major contributor to treatment response with both chemotherapy and radiotherapy (14). This is one of the reasons why treatment time (e.g. duration of radiotherapy) is thought to be very important (15). In a recent review Whitfield et al. (16) showed that proliferation may underlie the predictive power of many previously identified signatures. Whitfield et al. (16) showed that in almost every supervised derived signature a large subset of genes involved in proliferation is included (4, 17-20). In some cases, these classifiers have even been designated as ‘proliferation’ signatures although there derivation was not based on this phenotype. Two of these signatures have recently made it to the clinical setting as a diagnostic tool for patients with breast cancer (11, 21). Based on these results, it is hypothesized that derivation of a specific in vitro derived proliferation signature derived from gene expression data would provide more valuable information on tumor status, prognosis and prediction.

In view of the above, it is apparent that there exists a need for improved proliferation signatures.

### SUMMARY

In one aspect, the present invention provides for methods for predicting patient response to cancer treatment comprising measuring in a biological sample from a patient the levels of gene expression of a plurality of genes selected from the groups consisting of Group A, B, C, D, E, F, G, H, I, J, and K, defined below: Group A: Genes corresponding to transcripts associated with the Unigene ID Nos. Hs.121025, Hs.126714, Hs.132966, Hs.141125, Hs.156346, Hs.184339, Hs.1973, Hs.270845, Hs.294088, Hs.300701, Hs.308045, Hs.334562, Hs.339665, Hs.369279, Hs.405925, Hs.418533, Hs.433615, Hs.434250, Hs.435570, Hs.436912, Hs.438550, Hs.446017, Hs.472716, Hs.477879, Hs.503749, Hs.522632, Hs.524571, Hs.532968, Hs.533059, Hs.535012, Hs.58992, Hs.591697, Hs.603315, Hs.613351, Hs.642598, Hs.656, Hs.75318, Hs.88523, Hs.89497, Hs.93002, Hs.615092, Hs.62180, Hs.532803, Hs.240, Hs.444028, Hs.58974, Hs.104019, Hs.1594, Hs.178695, Hs.183800, Hs.194698, Hs.20575, Hs.226755, Hs.234545, Hs.239, Hs.244580, Hs.250822, Hs.28465, Hs.368710, Hs.374378, Hs.386189, Hs.436187, Hs.469649, Hs.476306, Hs.482233, Hs.497741, Hs.506652, Hs.509008, Hs.514033, Hs.514527, Hs.592049, Hs.592116, Hs.593658, Hs.631699, Hs.631750, Hs.644048, Hs.72550, Hs.75066, Hs.77695, Hs.83758, Hs.152385, Hs.165607, Hs.203965, Hs.208912, Hs.226390, Hs.26516, Hs.35086, Hs.368563, Hs.403171, Hs.409065, Hs.434886, Hs.436341, Hs.444082, Hs.485640, Hs.498248, Hs.513126, Hs.5199, Hs.520943, Hs.534339, Hs.558393, Hs.567267, Hs.575032, Hs.591046, Hs.591322, Hs.592338, Hs.81892, Hs.83765, Hs.88663, Hs.99480, and Hs.484950; b. Group B: Genes corresponding to transcripts associated with the Unigene ID Nos. Hs.524571, Hs.226390, Hs.436187, Hs.472716, and Hs.194698; c. Group C: Genes corresponding to transcripts associated with the Unigene ID Nos. Hs.368563, Hs.444028, Hs.58992, Hs.575032, and Hs.591697; d. Group D: Genes corresponding to transcripts associated with the Unigene ID Nos. Hs.436187, Hs.194698, Hs.250822, Hs.93002, and Hs.308045; e. Group E: Genes corresponding to transcripts associated with the Unigene ID Nos. Hs.58992, Hs.522632, Hs.446017, Hs.240, and Hs.533059; f. Group F: Genes corresponding to transcripts associated with the Unigene ID Nos. Hs.58974, Hs.75318, Hs.506652, Hs.184339, and Hs.81892; g. Group G: Genes corresponding to transcripts associated with the Unigene ID Nos. Hs.524571, Hs.226390, Hs.436187, Hs.472716, Hs.194698, Hs.386189, Hs.409065, Hs.5199, Hs.434250, and Hs.93002; h. Group H: Genes corresponding to transcripts associated with the Unigene ID Nos. Hs.368563, Hs.444028, Hs.58992, Hs.575032, Hs.591697, Hs.631750, Hs.250822, Hs.77695, Hs.194698, and Hs.631699; i. Group I: Genes corresponding to transcripts associated with the Unigene ID Nos. Hs.436187, Hs.194698, Hs.250822, Hs.93002, Hs.308045, Hs.444082, Hs.1594, Hs.184339, Hs.5199, and Hs.409065; j. Group J: Genes corresponding to transcripts associated with the Unigene ID Nos. Hs.58992, Hs.522632, Hs.446017, Hs.240, Hs.533059, Hs.513126, Hs.132966, Hs.532803, Hs.239, and Hs.58974; and k. Group K: Genes corresponding to transcripts associated with the Unigene ID Nos. Hs.58974, Hs.75318, Hs.506652, Hs.184339, Hs.81892, Hs.591322, Hs.156346, Hs.72550, Hs.374378, and Hs.77695; creating a signature score from said levels of gene expression; and correlating the signature score with a predicted response to cancer treatment.

In certain embodiments, the levels of gene expression are measured by determining the levels of expression of a group of polynucleotide sequences selected from the group consisting of: l. the sequences SEQ ID NOS: 1-110; m. the sequences SEQ ID NOS: 27, 85, 62, 23, and 51; n. the sequences SEQ ID NOS: 88, 45, 31, 102, and 32; o. the sequences SEQ ID NOS: 62, 51, 57, 40, and 11; p. the sequences SEQ ID NOS: 31, 26, 22, 44, and 29; q. the sequences SEQ ID NOS: 46, 37, 67, 6, and 106; r. the sequences SEQ ID NOS: 27, 85, 62, 23, 51, 61, 90, 97, 18, and 40; s. the sequences SEQ ID NOS: 88, 45, 31, 102, 32, 75, 57, 79, 51, and 74; t. the sequences SEQ ID NOS: 62, 51, 57, 40, 11, 93, 48, 6, 97, and 90; u. the sequences SEQ ID NOS: 31, 26, 22, 44, 29, 96, 3, 43, 55, and 46; and v. the sequences SEQ ID NOS: 46, 37, 67, 6, 106, 104, 5, 77, 60, and 79. In particular embodiments, the cancer is breast, renal, or lung cancer. In certain embodiments, the measuring of the levels of gene expression is carried out on RNA from said biological sample. The biological sample in particular embodiments is from a tumor, a cancerous tissue, a pre-cancerous tissue, a biopsy, a tissue, lymph node, a surgical excision, blood, serum, urine, an organ, or saliva. The treatment of the cancer may comprise radiotherapy, fractionated radiotherapy, chemotherapy, or chemo-radiotherapy in particular embodiments.

In a second aspect, the present invention provides for microarrays comprising: a solid substrate and a plurality of nucleic acid probes capable of detecting the levels of gene expression of a plurality of genes selected from the groups consisting of Group A, B, C, D, E, F, G, H, I, J, and K, defined below: a. Group A: Genes corresponding to transcripts associated with the Unigene ID Nos. Hs.121025, Hs.126714, Hs.132966, Hs.141125, Hs.156346, Hs.184339, Hs.1973, Hs.270845, Hs.294088, Hs.300701, Hs.308045, Hs.334562, Hs.339665, Hs.369279, Hs.405925, Hs.418533, Hs.433615, Hs.434250, Hs.435570, Hs.436912, Hs.438550, Hs.446017, Hs.472716, Hs.477879, Hs.503749, Hs.522632, Hs.524571, Hs.532968, Hs.533059, Hs.535012, Hs.58992, Hs.591697, Hs.603315, Hs.613351, Hs.642598, Hs.656, Hs.75318, Hs.88523, Hs.89497, Hs.93002, Hs.615092, Hs.62180, Hs.532803, Hs.240, Hs.444028, Hs.58974, Hs.104019, Hs.1594, Hs.178695, Hs.183800, Hs.194698, Hs.20575, Hs.226755, Hs.234545, Hs.239, Hs.244580, Hs.250822, Hs.28465, Hs.368710, Hs.374378, Hs.386189, Hs.436187, Hs.469649, Hs.476306, Hs.482233, Hs.497741, Hs.506652, Hs.509008, Hs.514033, Hs.514527, Hs.592049, Hs.592116, Hs.593658, Hs.631699, Hs.631750, Hs.644048, Hs.72550, Hs.75066, Hs.77695, Hs.83758, Hs.152385, Hs.165607, Hs.203965, Hs.208912, Hs.226390, Hs.26516, Hs.35086, Hs.368563, Hs.403171, Hs.409065, Hs.434886, Hs.436341, Hs.444082, Hs.485640, Hs.498248, Hs.513126, Hs.5199, Hs.520943, Hs.534339, Hs.558393, Hs.567267, Hs.575032, Hs.591046, Hs.591322, Hs.592338, Hs.81892, Hs.83765, Hs.88663, Hs.99480, and Hs.484950; b. Group B: Genes corresponding to transcripts associated with the Unigene ID Nos. Hs.524571, Hs.226390, Hs.436187, Hs.472716, and Hs.194698; c. Group C: Genes corresponding to transcripts associated with the Unigene ID Nos. Hs.368563, Hs.444028, Hs.58992, Hs.575032, and Hs.591697; d. Group D: Genes corresponding to transcripts associated with the Unigene ID Nos. Hs.436187, Hs.194698, Hs.250822, Hs.93002, and Hs.308045; e. Group E: Genes corresponding to transcripts associated with the Unigene ID Nos. Hs.58992, Hs.522632, Hs.446017, Hs.240, and Hs.533059; f. Group F: Genes corresponding to transcripts associated with the Unigene ID Nos. Hs.58974, Hs.75318, Hs.506652, Hs.184339, and Hs.81892; g. Group G: Genes corresponding to transcripts associated with the Unigene ID Nos. Hs.524571, Hs.226390, Hs.436187, Hs.472716, Hs.194698, Hs.386189, Hs.409065, Hs.5199, Hs.434250, and Hs.93002; h. Group H: Genes corresponding to transcripts associated with the Unigene ID Nos. Hs.368563, Hs.444028, Hs.58992, Hs.575032, Hs.591697, Hs.631750, Hs.250822, Hs.77695, Hs.194698, and Hs.631699; i. Group I: Genes corresponding to transcripts associated with the Unigene ID Nos. Hs.436187, Hs.194698, Hs.250822, Hs.93002, Hs.308045, Hs.444082, Hs.1594, Hs.184339, Hs.5199, and Hs.409065; j. Group J: Genes corresponding to transcripts associated with the Unigene ID Nos. Hs.58992, Hs.522632, Hs.446017, Hs.240, Hs.533059, Hs.513126, Hs.132966, Hs.532803, Hs.239, and Hs.58974; and k. Group K: Genes corresponding to transcripts associated with the Unigene ID Nos. Hs.58974, Hs.75318, Hs.506652, Hs.184339, Hs.81892, Hs.591322, Hs.156346, Hs.72550, Hs.374378, and Hs.77695. In particular embodiments, the microarray contains a plurality of nucleic acid probes are capable of detecting the expression of a group of sequences selected from the group consisting of: l. the sequences SEQ ID NOS: 1-110; m. the sequences SEQ ID NOS: 27, 85, 62, 23, and 51; n. the sequences SEQ ID NOS: 88, 45, 31, 102, and 32; o. the sequences SEQ ID NOS: 62, 51, 57, 40, and 11; p. the sequences SEQ ID NOS: 31, 26, 22, 44, and 29; q. the sequences SEQ ID NOS: 46, 37, 67, 6, and 106; r. the sequences SEQ ID NOS: 27, 85, 62, 23, 51, 61, 90, 97, 18, and 40; s. the sequences SEQ ID NOS: 88, 45, 31, 102, 32, 75, 57, 79, 51, and 74; t. the sequences SEQ ID NOS: 62, 51, 57, 40, 11, 93, 48, 6, 97, and 90; u. the sequences SEQ ID NOS: 31, 26, 22, 44, 29, 96, 3, 43, 55, and 46; and v. the sequences SEQ ID NOS: 46, 37, 67, 6, 106, 104, 5, 77, 60, and 79. In particular embodiments, the plurality of probes comprise DNA sequences. The plurality of probes are capable of hybridizing to the sequences of at least one of the groups (l)-(v) under the hybridization conditions of 6×SSC at 65° C., in certain embodiments. In certain embodiments, the plurality of probes each comprise from about 15 to 50 base pairs of DNA.

In a third aspect, the present invention provides for kits comprising a microarray comprising a plurality of nucleic acid probes capable of detecting the expression of a group of sequences selected from the group consisting of: groups (l)-(v) described above; and directions for use of the kit.

In a fourth aspect, the present invention provides for methods of treating cancer comprising measuring in a biological sample from a patient the levels of gene expression of a plurality of genes selected from the groups consisting of Group A, B, C, D, E, F, G, H, I, J, and K, defined below: a. Group A: Genes corresponding to transcripts associated with the Unigene ID Nos. Hs.121025, Hs.126714, Hs.132966, Hs.141125, Hs.156346, Hs.184339, Hs.1973, Hs.270845, Hs.294088, Hs.300701, Hs.308045, Hs.334562, Hs.339665, Hs.369279, Hs.405925, Hs.418533, Hs.433615, Hs.434250, Hs.435570, Hs.436912, Hs.438550, Hs.446017, Hs.472716, Hs.477879, Hs.503749, Hs.522632, Hs.524571, Hs.532968, Hs.533059, Hs.535012, Hs.58992, Hs.591697, Hs.603315, Hs.613351, Hs.642598, Hs.656, Hs.75318, Hs.88523, Hs.89497, Hs.93002, Hs.615092, Hs.62180, Hs.532803, Hs.240, Hs.444028, Hs.58974, Hs.104019, Hs.1594, Hs.178695, Hs.183800, Hs.194698, Hs.20575, Hs.226755, Hs.234545, Hs.239, Hs.244580, Hs.250822, Hs.28465, Hs.368710, Hs.374378, Hs.386189, Hs.436187, Hs.469649, Hs.476306, Hs.482233, Hs.497741, Hs.506652, Hs.509008, Hs.514033, Hs.514527, Hs.592049, Hs.592116, Hs.593658, Hs.631699, Hs.631750, Hs.644048, Hs.72550, Hs.75066, Hs.77695, Hs.83758, Hs.152385, Hs.165607, Hs.203965, Hs.208912, Hs.226390, Hs.26516, Hs.35086, Hs.368563, Hs.403171, Hs.409065, Hs.434886, Hs.436341, Hs.444082, Hs.485640, Hs.498248, Hs.513126, Hs.5199, Hs.520943, Hs.534339, Hs.558393, Hs.567267, Hs.575032, Hs.591046, Hs.591322, Hs.592338, Hs.81892, Hs.83765, Hs.88663, Hs.99480, and Hs.484950; b. Group B: Genes corresponding to transcripts associated with the Unigene ID Nos. Hs.524571, Hs.226390, Hs.436187, Hs.472716, and Hs.194698; c. Group C: Genes corresponding to transcripts associated with the Unigene ID Nos. Hs.368563, Hs.444028, Hs.58992, Hs.575032, and Hs.591697; d. Group D: Genes corresponding to transcripts associated with the Unigene ID Nos. Hs.436187, Hs.194698, Hs.250822, Hs.93002, and Hs.308045; e. Group E: Genes corresponding to transcripts associated with the Unigene ID Nos. Hs.58992, Hs.522632, Hs.446017, Hs.240, and Hs.533059; f. Group F: Genes corresponding to transcripts associated with the Unigene ID Nos. Hs.58974, Hs.75318, Hs.506652, Hs.184339, and Hs.81892; g. Group G: Genes corresponding to transcripts associated with the Unigene ID Nos. Hs.524571, Hs.226390, Hs.436187, Hs.472716, Hs.194698, Hs.386189, Hs.409065, Hs.5199, Hs.434250, and Hs.93002; h. Group H: Genes corresponding to transcripts associated with the Unigene ID Nos. Hs.368563, Hs.444028, Hs.58992, Hs.575032, Hs.591697, Hs.631750, Hs.250822, Hs.77695, Hs.194698, and Hs.631699; i. Group I: Genes corresponding to transcripts associated with the Unigene ID Nos. Hs.436187, Hs.194698, Hs.250822, Hs.93002, Hs.308045, Hs.444082, Hs.1594, Hs.184339, Hs.5199, and Hs.409065; j. Group J: Genes corresponding to transcripts associated with the Unigene ID Nos. Hs.58992, Hs.522632, Hs.446017, Hs.240, Hs.533059, Hs.513126, Hs.132966, Hs.532803, Hs.239, and Hs.58974; and k. Group K: Genes corresponding to transcripts associated with the Unigene ID Nos. Hs.58974, Hs.75318, Hs.506652, Hs.184339, Hs.81892, Hs.591322, Hs.156346, Hs.72550, Hs.374378, and Hs.77695; and administering a therapeutically effective amount of one or more cancer treatment agents selected from the group consisting of: cancer chemotherapeutic agents and radiation; or performing surgery on the patient; or a combination thereof. In further embodiments, the levels of gene expression are measured by determining the levels of expression of a group of polynucleotide sequences selected from the group consisting of groups (l)-(v) described above. In certain embodiments, the one or more cancer treatment agents are selected from the group consisting of: paclitaxel, docetaxel, imatinib mesylate, sunitinib malate, cisplatin, etoposide, vinblastine, methotrexate, adriamycin, cyclophosphamide, doxorubicin, daunomycin, 5-fluoruracil, vincristine, endostatin, angiostatin, bevacizumab, and rituximab. In another embodiment, the one or more cancer treatment agents is radiation. In particular embodiments, the cancer being treated is breast, renal, or lung cancer. In certain embodiments, the methods of treatment comprise surgery.

Further objects, features and advantages of this invention will become readily apparent to persons skilled in the art after a review of the following description, with reference to the drawings and claims that are appended to and form a part of this specification.

### BRIEF DESCRIPTION OF THE DRAWINGS

FIG. 1 depicts Kaplan-Meier survival curves for proliferation signature (a) on various datasets (A: Miller dataset, B: Wang dataset, C: van de Vijver dataset, D: Zhao dataset, E: Beer dataset). A signature score was calculated for each patient in the different datasets. These scores were used to cluster the patients in two groups, one with low expression and one with high expression of the signature. Kaplan-Meier survival curves for the two groups were compared. Patients with tumors with a high proliferation signature score had worse outcomes than those with tumors with a low proliferation signature score. The Kaplan-Meier survival curve with the low signature score is represented by a solid line, and in each panel is the top curve. The Kaplan-Meier survival curve with the high signature score is represented by a dotted line, and in each panel is the lower curve;

FIG. 2 depicts ROC curves of model of clinical factors with and without proliferation signature (A: Miller dataset, B: van de Vijver dataset, C: Zhao dataset). A model of the clinical factors with and without the signature was generated. Receiver operator curves (ROC) were used to compare the two models in three datasets. Inclusion of the proliferation signature in the model increased the prediction performance in two datasets. The model with the proliferation signature is represented by a dotted line. The model without the proliferation signature is the solid line. The models with the proliferation signature in Panels A, B, and C have an AUC of 0.75, 0.76, and 0.87, respectively. The models with the proliferation signature in Panels A, B, and C have an AUC of 0.72, 0.73, and 0.88, respectively;

FIG. 3 depicts Kaplan-Meier curves for signature (b) run on the Miller data set (the top curve is the low signature score, the bottom curve is the high signature score);

FIG. 4 depicts Kaplan-Meier curves for signature (c) run on the Wang data set (the top curve is the low signature score, the bottom curve is the high signature score);

FIG. 5 depicts Kaplan-Meier curves for signature (d) run on the van de Vijver data set (the top curve is the low signature score, the bottom curve is the high signature score);

FIG. 6 depicts Kaplan-Meier curves for signature (e) run on the Zhao data set (the top curve is the low signature score, the bottom curve is the high signature score);

FIG. 7 depicts Kaplan-Meier curves for signature (f) run on the Beer data set (the top curve is the low signature score, the bottom curve is the high signature score);

FIG. 8 depicts Kaplan-Meier curves for signature (g) run on the Miller data set (the top curve is the low signature score, the bottom curve is the high signature score);

FIG. 9 depicts Kaplan-Meier curves for signature (h) run on the Wang data set (the top curve is the low signature score, the bottom curve is the high signature score);

FIG. 10 depicts Kaplan-Meier curves for signature (i) run on the van de Vijver data set (the top curve is the low signature score, the bottom curve is the high signature score);

FIG. 11 depicts Kaplan-Meier curves for signature (j) run on the Zhao data set (the top curve is the low signature score, the bottom curve is the high signature score); and

FIG. 12 depicts Kaplan-Meier curves for signature (k) run on the Beer data set (the top curve is the low signature score, the bottom curve is the high signature score).

### DETAILED DESCRIPTION

Tumor proliferation is one of the main biological phenotypes limiting cure in oncology. Extensive research, including microarray experiments, is being performed to unravel the key-players in this process. To exploit the potential of published gene expression data, creation of a signature for proliferation can provide valuable information on tumor status, prognosis and prediction. This will help individualizing treatment and should result in better tumor control, and more rapid and cost-effective research and development.

The present invention provides methods and compositions for predicting patient response to cancer treatment using gene signatures. The methods typically involve measuring in a biological sample from a patient the levels of gene expression of a group of the genes corresponding to transcripts associated with a particular groups of Unigene ID Nos. In particular embodiments, the Unigene ID Nos. are selected from groups (a)-(k) as set out above. One Unigene ID No. may have multiple transcripts associated with it. Examples of a DNA sequence associated with each Unigene ID No. of groups (a)-(k) may be found in Table 1 as SEQ ID NOS. 1-110:

TABLE 1
Proliferation Signature

| | | Cell | | | |
|---|---|---|---|---|---|
| SEQ | | cycle | | | |
| ID NO: | UnigeneID | phase | Weight | Symbol | Name |
| 1 | Hs.121025 | G2 | 1 | ZNHIT2 | Zinc finger, HIT type 2 |
| 2 | Hs.126714 | G2 | 1 | CIITA | Class II, major |
| | | | | | histocompatibility complex, |
| | | | | | transactivator |
| 3 | Hs.132966 | G2 | 1 | MET | Met proto-oncogene |
| | | | | | (hepatocyte growth factor |
| | | | | | receptor) |
| 4 | Hs.141125 | G2 | 1 | CASP3 | Caspase 3, apoptosis- |
| | | | | | related cysteine peptidase |
| 5 | Hs.156346 | G2 | 1 | TOP2A | Topoisomerase (DNA) II |
| | | | | | alpha 170 kDa |
| 6 | Hs.184339 | G2 | 1 | MELK | Maternal embryonic |
| | | | | | leucine zipper kinase |
| 7 | Hs.1973 | G2 | 1 | CCNF | Cyclin F |
| 8 | Hs.270845 | G2 | 1 | KIF23 | Kinesin family member 23 |
| 9 | Hs.294088 | G2 | 1 | MND1 | Meiotic nuclear divisions 1 |
| | | | | | homolog (S. cerevisiae) |
| 10 | Hs.300701 | G2 | 1 | TUBB2B | Tubulin, beta 2B |
| 11 | Hs.308045 | G2 | 1 | NCAPH | Non-SMC condensin I |
| | | | | | complex, subunit H |
| 12 | Hs.334562 | G2 | 1 | CDC2 | Cell division cycle 2, G1 to |
| | | | | | S and G2 to M |
| 13 | Hs.339665 | G2 | 1 | LOC653820 | Similar to family with |
| | | | | | sequence similarity 72, |
| | | | | | member A |
| 14 | Hs.369279 | G2 | 1 | NLRP2 | NLR family, pyrin domain |
| | | | | | containing 2 |
| 15 | Hs.405925 | G2 | 1 | PSRC1 | Proline/serine-rich coiled- |
| | | | | | coil 1 |
| 16 | Hs.418533 | G2 | 1 | BUB3 | BUB3 budding uninhibited |
| | | | | | by benzimidazoles 3 |
| | | | | | homolog (yeast) |
| 17 | Hs.433615 | G2 | 1 | TUBB2C | Tubulin, beta 2C |
| 18 | Hs.434250 | G2 | 1 | CKAP2L | Cytoskeleton associated |
| | | | | | protein 2-like |
| 19 | Hs.435570 | G2 | 1 | CDKL5 | Cyclin-dependent kinase- |
| | | | | | like 5 |
| 20 | Hs.436912 | G2 | 1 | KIFC1 | Kinesin family member C1 |
| 21 | Hs.438550 | G2 | 1 | NCAPD3 | Non-SMC condensin II |
| | | | | | complex, subunit D3 |
| 22 | Hs.446017 | G2 | 1 | WSB1 | WD repeat and SOCS box- |
| | | | | | containing 1 |
| 23 | Hs.472716 | G2 | 1 | FAM83D | Family with sequence |
| | | | | | similarity 83, member D |
| 24 | Hs.477879 | G2 | 1 | H2AFX | H2A histone family, |
| | | | | | member X |
| 25 | Hs.503749 | G2 | 1 | H2- | Alpha-tubulin isotype H2- |
| | | | | ALPHA | alpha |
| 26 | Hs.522632 | G2 | 1 | TIMP1 | TIMP metallopeptidase |
| | | | | | inhibitor 1 |
| 27 | Hs.524571 | G2 | 1 | CDCA8 | Cell division cycle |
| | | | | | associated 8 |
| 28 | Hs.532968 | G2 | 1 | DKFZp762E1312 | Hypothetical protein |
| | | | | | DKFZp762E1312 |
| 29 | Hs.533059 | G2 | 1 | TUBB | Tubulin, beta |
| 30 | Hs.535012 | G2 | 1 | LOC441052 | Hypothetical gene |
| | | | | | supported by AF131741 |
| 31 | Hs.58992 | G2 | 1 | SMC4 | Structural maintenance of |
| | | | | | chromosomes 4 |
| 32 | Hs.591697 | G2 | 1 | MAD2L1 | MAD2 mitotic arrest |
| | | | | | deficient-like 1 (yeast) |
| 33 | Hs.603315 | G2 | 1 | | Transcribed locus |
| 34 | Hs.613351 | G2 | 1 | KIF22 | Kinesin family member 22 |
| 35 | Hs.642598 | G2 | 1 | ZNF587 | Zinc finger protein 587 |
| 36 | Hs.656 | G2 | 1 | CDC25C | Cell division cycle 25 |
| | | | | | homolog C (S. pombe) |
| 37 | Hs.75318 | G2 | 1 | TUBA4A | Tubulin, alpha 1 |
| 38 | Hs.88523 | G2 | 1 | C13orf3 | Chromosome 13 open |
| | | | | | reading frame 3 |
| 39 | Hs.89497 | G2 | 1 | LMNB1 | Lamin B1 |
| 40 | Hs.93002 | G2 | 1 | UBE2C | Ubiquitin-conjugating |
| | | | | | enzyme E2C |
| 41 | Hs.615092 | G2 | 1 | NUSAP1 | Nucleolar and spindle |
| | | G2/M | | | associated protein 1 |
| 42 | Hs.62180 | G2 | 1 | ANLN | Anillin, actin binding protein |
| | | G2/M | | | |
| 43 | Hs.532803 | G2 | 1 | HN1 | Hematological and |
| | | G2/M | | | neurological expressed 1 |
| 44 | Hs.240 | G2 | 1 | MPHOSPH1 | M-phase phosphoprotein 1 |
| | | G2/M | | | |
| 45 | Hs.444028 | G2 | 1 | CKAP2 | Cytoskeleton associated |
| | | G2/M | | | protein 2 |
| 46 | Hs.58974 | G2 | 1 | CCNA2 | Cyclin A2 |
| | | G2/M | | | |
| 47 | Hs.104019 | G2/M | 1 | TACC3 | Transforming, acidic |
| | | | | | coiled-coil containing |
| | | | | | protein 3 |
| 48 | Hs.1594 | G2/M | 1 | CENPA | Centromere protein A |
| 49 | Hs.178695 | G2/M | 1 | MAPK13 | Mitogen-activated protein |
| | | | | | kinase 13 |
| 50 | Hs.183800 | G2/M | 1 | RANGAP1 | Ran GTPase activating |
| | | | | | protein 1 |
| 51 | Hs.194698 | G2/M | 1 | CCNB2 | Cyclin B2 |
| 52 | Hs.20575 | G2/M | 1 | GAS2L3 | Growth arrest-specific 2 |
| | | | | | like 3 |
| 53 | Hs.226755 | G2/M | 1 | YWHAH | Tyrosine 3- |
| | | | | | monooxygenase/tryptophan |
| | | | | | 5-monooxygenase |
| | | | | | activation protein, eta |
| | | | | | polypeptide |
| 54 | Hs.234545 | G2/M | 1 | NUF2 | NUF2, NDC80 kinetochore |
| | | | | | complex component, |
| | | | | | homolog (S. cerevisiae) |
| 55 | Hs.239 | G2/M | 1 | FOXM1 | Forkhead box M1 |
| 56 | Hs.244580 | G2/M | 1 | TPX2 | TPX2, microtubule- |
| | | | | | associated, homolog |
| | | | | | (Xenopus laevis) |
| 57 | Hs.250822 | G2/M | 1 | AURKA | Aurora kinase A |
| 58 | Hs.28465 | G2/M | 1 | RP11- | Similar to RIKEN cDNA |
| | | | | 11C5.2 | 2410129H14 |
| 59 | Hs.368710 | G2/M | 1 | CCDC99 | Coiled-coil domain |
| | | | | | containing 99 |
| 60 | Hs.374378 | G2/M | 1 | CKS1B | CDC28 protein kinase |
| | | | | | regulatory subunit 1B |
| 61 | Hs.386189 | G2/M | 1 | GTSE1 | G-2 and S-phase |
| | | | | | expressed 1 |
| 62 | Hs.436187 | G2/M | 1 | TRIP13 | Thyroid hormone receptor |
| | | | | | interactor 13 |
| 63 | Hs.469649 | G2/M | 1 | BUB1 | BUB1 budding uninhibited |
| | | | | | by benzimidazoles 1 |
| | | | | | homolog (yeast) |
| 64 | Hs.476306 | G2/M | 1 | WDR51A | WD repeat domain 51A |
| 65 | Hs.482233 | G2/M | 1 | DEPDC1B | DEP domain containing 1B |
| 66 | Hs.497741 | G2/M | 1 | CENPF | Centromere protein F, |
| | | | | | 350/400ka (mitosin) |
| 67 | Hs.506652 | G2/M | 1 | PWP1 | PWP1 homolog (S. cerevisiae) |
| 68 | Hs.509008 | G2/M | 1 | KIAA1333 | KIAA1333 |
| 69 | Hs.514033 | G2/M | 1 | SPAG5 | Sperm associated antigen 5 |
| 70 | Hs.514527 | G2/M | 1 | BIRC5 | Baculoviral IAP repeat- |
| | | | | | containing 5 (survivin) |
| 71 | Hs.592049 | G2/M | 1 | PLK1 | Polo-like kinase 1 |
| | | | | | (Drosophila) |
| 72 | Hs.592116 | G2/M | 1 | FAM64A | Family with sequence |
| | | | | | similarity 64, member A |
| 73 | Hs.593658 | G2/M | 1 | | Transcribed locus |
| 74 | Hs.631699 | G2/M | 1 | BUB1B | BUB1 budding uninhibited |
| | | | | | by benzimidazoles 1 |
| | | | | | homolog beta (yeast) |
| 75 | Hs.631750 | G2/M | 1 | PRR11 | Proline rich 11 |
| 76 | Hs.644048 | G2/M | 1 | | Transcribed locus |
| 77 | Hs.72550 | G2/M | 1 | HMMR | Hyaluronan-mediated |
| | | | | | motility receptor (RHAMM) |
| 78 | Hs.75066 | G2/M | 1 | TSN | Translin |
| 79 | Hs.77695 | G2/M | 1 | DLG7 | Discs, large homolog 7 |
| | | | | | (Drosophila) |
| 80 | Hs.83758 | G2/M | 1 | CKS2 | CDC28 protein kinase |
| | | | | | regulatory subunit 2 |
| 81 | Hs.152385 | S | 1 | KIAA1370 | KIAA1370 |
| | | phase | | | |
| 82 | Hs.165607 | S | 1 | FLJ25416 | Hypothetical protein |
| | | phase | | | FLJ25416 |
| 83 | Hs.203965 | S | 1 | PHTF2 | Putative homeodomain |
| | | phase | | | transcription factor 2 |
| 84 | Hs.208912 | S | 1 | CENPM | Centromere protein M |
| | | phase | | | |
| 85 | Hs.226390 | S | 1 | RRM2 | Ribonucleotide reductase |
| | | phase | | | M2 polypeptide |
| 86 | Hs.26516 | S | 1 | ASF1B | ASF1 anti-silencing |
| | | phase | | | function 1 homolog B (S. cerevisiae) |
| 87 | Hs.35086 | S | 1 | USP1 | Ubiquitin specific peptidase 1 |
| | | phase | | | |
| 88 | Hs.368563 | S | 1 | ABCC5 | ATP-binding cassette, sub- |
| | | phase | | | family C (CFTR/MRP), |
| | | | | | member 5 |
| 89 | Hs.403171 | S | 1 | EFHC1 | EF-hand domain (C- |
| | | phase | | | terminal) containing 1 |
| 90 | Hs.409065 | S | 1 | FEN1 | Flap structure-specific |
| | | phase | | | endonuclease 1 |
| 91 | Hs.434886 | S | 1 | CDCA5 | Cell division cycle |
| | | phase | | | associated 5 |
| 92 | Hs.436341 | S | 1 | DONSON | Downstream neighbor of |
| | | phase | | | SON |
| 93 | Hs.444082 | S | 1 | EZH2 | Enhancer of zeste homolog |
| | | phase | | | 2 (Drosophila) |
| 94 | Hs.485640 | S | 1 | PRIM2A | Primase, polypeptide 2A, |
| | | phase | | | 58 kDa |
| 95 | Hs.498248 | S | 1 | EXO1 | Exonuclease 1 |
| | | phase | | | |
| 96 | Hs.513126 | S | 1 | KIAA1794 | KIAA1794 |
| | | phase | | | |
| 97 | Hs.5199 | S | 1 | UBE2T | Ubiquitin-conjugating |
| | | phase | | | enzyme E2T (putative) |
| 98 | Hs.520943 | S | 1 | RFC2 | Replication factor C |
| | | phase | | | (activator 1) 2, 40 kDa |
| 99 | Hs.534339 | S | 1 | PRIM1 | Primase, polypeptide 1, |
| | | phase | | | 49 kDa |
| 100 | Hs.558393 | S | 1 | RRM1 | Ribonucleotide reductase |
| | | phase | | | M1 polypeptide |
| 101 | Hs.567267 | S | 1 | FANCA | Fanconi anemia, |
| | | phase | | | complementation group A |
| 102 | Hs.575032 | S | 1 | MLF1IP | MLF1 interacting protein |
| | | phase | | | |
| 103 | Hs.591046 | S | 1 | RAD51AP1 | RAD51 associated protein 1 |
| | | phase | | | |
| 104 | Hs.591322 | S | 1 | RFC4 | Replication factor C |
| | | phase | | | (activator 1) 4, 37 kDa |
| 105 | Hs.592338 | S | 1 | TYMS | Thymidylate synthetase |
| | | phase | | | |
| 106 | Hs.81892 | S | 1 | CSNK1G1 | Casein kinase 1, gamma 1 |
| | | phase | | | |
| 107 | Hs.83765 | S | 1 | DHFR | Dihydrofolate reductase |
| | | phase | | | |
| 108 | Hs.88663 | S | 1 | CENPQ | Centromere protein Q |
| | | phase | | | |
| 109 | Hs.99480 | S | 1 | ESCO2 | Establishment of cohesion |
| | | phase | | | 1 homolog 2 (S. cerevisiae) |
| 110 | Hs.484950 | *S | 1 | HIST1H2AC | Histone cluster 1, H2ac |
| | | phase | | | |

The levels of gene expression may also be measured by determining the levels of expression of a group of polynucleotide sequences that are members of a signature. Examples of DNA sequences of associated with a signature include any of groups (k)-(v). Thus, examples of signatures include group (l): SEQ ID NOS: 1-110; group m. the sequences SEQ ID NOS: 27, 85, 62, 23, and 51; group (n). the sequences SEQ ID NOS: 88, 45, 31, 102, and 32; group (o) the sequences SEQ ID NOS: 62, 51, 57, 40, and 11; group (p) the sequences SEQ ID NOS: 31, 26, 22, 44, and 29; group (q) the sequences SEQ ID NOS: 46, 37, 67, 6, and 106; group (r) the sequences SEQ ID NOS: 27, 85, 62, 23, 51, 61, 90, 97, 18, and 40; group (s) SEQ ID NOS: 88, 45, 31, 102, 32, 75, 57, 79, 51, and 74; group (t) SEQ ID NOS: 62, 51, 57, 40, 11, 93, 48, 6, 97, and 90; group (u) SEQ ID NOS: 31, 26, 22, 44, 29, 96, 3, 43, 55, and 46; and group (v) the sequences SEQ ID NOS: 46, 37, 67, 6, 106, 104, 5, 77, 60, and 79.

To examine the levels of gene expression of one or more sequences or Unigene ID Nos., a biological sample of a patient that is suffering from a cancer or who has yet to be diagnosed with cancer is typically assayed. A “biological sample” includes a sample from a tumor, cancerous tissue, pre-cancerous tissue, biopsy, tissue, lymph node, surgical excision, blood, serum, urine, organ, saliva, etc. obtained from a patient suffering from a cancer or who has yet to be diagnosed with cancer.

The biological sample is then typically assayed from the presence of one or more gene expression products such as RNA, cDNA, cRNA, protein, etc.

In one embodiment, mRNA from a biological sample is directly used in determining the levels of expression of a group of genes. In one particular embodiment, RNA is obtained from a biological sample. The RNA is then transformed into cDNA (complementary DNA) copy using methods known in the art. In particular embodiments, the cDNA is labeled with a fluorescent label or other detectable label. The cDNA is then hybridized to a substrate containing a plurality of probes of interest. A probe of interest typically hybridizes under stringent hybridization conditions to at least one DNA sequence of a gene signature. In certain embodiments, the plurality of probes are capable of hybridizing to the sequences of at least one of the group of DNA sequences of groups (l)-(v) under the hybridization conditions of 6×SSC (0.9 M NaCl, 0.09 M sodium citrate, pH 7.4) at 65° C. The probes may comprise nucleic acids. An example of a nucleic acid is DNA. The term “nucleic acid” refers to deoxyribonucleotides or ribonucleotides and polymers thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, peptide-nucleic acids (PNAs).

In certain cases, the probes will be from about 15 to about 50 base pairs in length. The amount of cDNA hybridization can be measured by assaying for the presence of the detectable label, such as a fluorophore. The quantification of the hybridization signal can be used to generate a score for a particular sequence or set of sequences in the gene signature for a particular patient or plurality of patients.

The term “detectable label” refers to a moiety that is attached through covalent or non-covalent means to an entity being measured or a probe. A “detectable label” can be a radioactive moiety, a fluorescent moiety, a chemiluminescent moiety, etc. The term “fluorescent label” refers to label that accepts radiant energy of one wavelength and emits radiant energy of a second wavelength. The presence of a detectable label may be assayed using methods known in the art that are appropriate to detect a particular label, such as spectrophotometric means (e.g., a spectrophotometer), radiometric means (e.g., scintillation counter), fluorometer, luminometer, etc.

Included within the scope of the invention are DNA microarrays containing a plurality of sequences that hybridize under stringent hybridization conditions to one or more of the gene sequences in a gene signature. An example of a substrate containing one or more probes of interest is a plurality of DNA probes that are affixed to a substrate. In certain embodiments, the substrate may comprise one or more materials such as gel, nitrocellulose, nylon, quartz, glass, metal, silica based materials, silica, resins, polymers, etc., or combinations thereof. Typically, the DNA probes comprise about 10-50 by of contiguous DNA. In certain embodiments, the DNA probes are from about 20 to about 50 by of contiguous DNA. In certain embodiments, the present invention relates to kits which comprising a microarray directions for its use. The kit may comprise a container which comprises one or more microarrays and directions for their use.

The biological sample may also be analyzed for gene expression of one or more genes in a signature using methods that can detect nucleic acids including, but not limited to, PCR (polymerase chain reaction); RT-PCT (reverse transcriptase-polymerase chain reaction); quantitative PCR, etc.

In certain embodiments, the levels of gene expression are measured by detecting the protein expression products of the genes or DNA sequences. The levels of protein products may be measured using methods known in the art including the use of antibodies which specifically bind to a particular protein. These antibodies, including polyclonal or monoclonal antibodies, may be produced using methods that are known in the art. These antibodies may also be coupled to a solid substrate to form an antibody chip or antibody microarray. Antibody or protein microarrays may be made using methods that are known in the art.

Once the levels of gene expression have been measured then a signature score is created. Examples of how to create a signature score are described herein. The signature score is then correlated with a predicted response to cancer treatment. Typically, a Kaplan-Meier curve may be generated to determine if the signature score is associated with a higher or lower survival rate. In particular embodiments, a positive or negative numerical weight may be assigned to a sequence or Unigene ID No. in the creation of a signature score. If the signature score is associated with a lower survival rate, then aggressive cancer treatment may be indicated. If the signature score is associated with a higher survival rate then less aggressive cancer treatment may be indicated.

The treatment of cancer in certain embodiments, involves measuring the levels of gene expression of a group of genes represented by Unigene ID Nos. selected from the group consisting of groups (a)-(k). The method of treatment typically further comprises administering a therapeutically effective amount of one or more cancer treatment agents selected from the group consisting of: cancer chemotherapeutic agents and radiation. The treatment of cancer may also comprise surgery or surgical procedures. The term “administering” refers to the method of contacting a compound with a subject. Modes of “administering” may include but are not limited to, methods that involve contacting the cancer chemotherapeutic agents intravenously, intraperitoneally, intranasally, transdermally, topically, via implantation, subcutaneously, parentally, intramuscularly, orally, systemically, and via adsorption. The term “treatment” includes the acute or prophylactic diminishment or alleviation of at least one symptom or characteristic associated or caused by the cancer being treated. For example, treatment can include diminishment of several symptoms of a cancer or complete eradication of a cancer. The phrase “therapeutically effective amount” means an amount of a cancer chemotherapeutic agent, or a pharmaceutically acceptable salt thereof, that is sufficient to inhibit, halt, or allow an improvement in the cancer being treated when administered alone or in conjunction with another pharmaceutical agent or treatment in a particular subject or subject population. For example in a human a therapeutically effective amount can be determined experimentally in a clinical setting, for the particular disease and subject being treated. It should be appreciated that determination of proper dosage forms, dosage amounts and routes of administration is within the level of ordinary skill in the pharmaceutical and medical arts.

It is within the purview of the skill medical practitioner to select an appropriate therapeutic regimen. Therapeutic regimens may be comprised of the use of cancer chemotherapeutic agents and/or radiation. A cancer chemotherapeutic agent is a chemical or biological agent (e.g., antibody, protein, RNA, DNA, etc.) that retards, slows, or stops the growth of cancer or is approved to treat cancer by the U.S. Food and Drug Administration. Examples of cancer chemotherapeutic agents include, but are not limited to: paclitaxel, docetaxel, imatinib mesylate, sunitinib malate, cisplatin, etoposide, vinblastine, methotrexate, adriamycin, cyclophosphamide, doxorubicin, daunomycin, 5-fluoruracil, vincristine, endostatin, angiostatin, bevacizumab, and rituximab. Another example of a cancer treatment agent is radiation. Thus, the cancer treatment may comprise radiotherapy, fractionated radiotherapy, chemotherapy, or chemo-radiotherapy (a combination of one or more chemotherapeutic agents and radiation). The cancer may be any type of cancer. In certain embodiments, the cancer is breast, renal, or lung cancer. Examples of cancer include, but are not limited to: small cell lung cancer, squamous cell lung carcinoma, glioma, breast cancer, prostate cancer, ovarian cancer, cervical cancer, gliobastoma, endometrial carcinoma, heptocellular carcinoma, colon cancer, lung cancer, melanoma, renal cell carcinoma, renal cancer, thyroid carcinoma, squamous cell lung carcinoma, leukemia, cell lymphoma, and lymphoproliferative disorders,

### Examples

Signature Score Methods

From in vitro published microarray studies, two proliferation signatures were compiled of 508 and 110 genes respectively. The prognostic value of these signatures was tested in five large clinical microarray datasets. More than 1,000 patients with breast, renal or lung cancer were included. A signature score was used to evaluate the performance of the signatures.

Results

One of the signatures (110 genes) (a signature that is also known as signature (a)—see Table 1 for listing of the 110 UniGene ID Nos.)) had significant prognostic value in all datasets. Stratifying patients in groups based on the signature score resulted in a clear difference in survival (p-values<0.05). Further multivariate Cox-regression analyses and AUC (area under the curve) calculations showed that this signature added substantial value to clinical factors used for prognosis and can be combined with other phenotype based signatures. In addition running 10,000 random gene sets showed the strength of the signature, no random signature showed significant results on all 5 datasets.

Conclusions

The proliferation signature is a strong prognostic factor, with the potential to be converted into a predictive test. It can be used to select patients who could benefit from accelerated radiotherapy or chemo-radiotherapy.

### Materials and Methods

Signature Derivation

From published microarray studies two different proliferation signatures were compiled. Whitfield et al. (22) studied the cell cycle in HeLa cells (cervix cancer cell line). Microarrays were performed on synchronized cell cultures at different time-points and genes that showed a periodic variation were selected (22). These genes were grouped according to the cell cycle phase in which their expression peaked. It is proposed that this gene set could be employed as a specific proliferation signature. Genes with a peak expression in G1 phase will represent non-proliferating cells and genes in S, G2 and M phase then represent proliferating cells. Another method to derive a proliferation signature with microarrays was employed by Chang et al. (6). Human fibroblasts were serum starved for 48 hours and then stimulated with serum to simulate a wound response. One of the most consistent and important effects in the serum response program is stimulation of proliferation. Abnormal proliferation is also a consistent characteristic of cancer cells, irrespective of a wound response (6). Chang et al. (6) therefore discarded the genes with a periodic behavior to specifically study the wound response. Here it is proposed that the set of genes discarded from the wound signature is a good representation of a proliferation signature. This signature is a subset of the signature derived from Whitfield et al. (22), however it is postulated that it is a better representative of proliferation and will be a better prognostic factor, since only this gene set shows a change in expression upon serum stimulation.

Datasets

Patient microarray and clinical follow-up data were collated to test the clinical value of the signatures. Datasets are publicly available in the microarray databases Gene Expression Omnibus (GEO: http://www.ncbi.nlm.nih.gov/projects/geo/) and Stanford Microarray Database (SMD: http://genome-www.stanford.edu/microarray) or elsewhere. Accessory clinical and followup data were also given or provided by the authors on request. In Table 2 an overview of the datasets and where they are accessible is provided:

TABLE 2
Overview of the analyzed patient microarray datasets

| | Cancer | Number of | |
|---|---|---|---|
| Dataset | type | patients | Source |
| Miller | Breast | 251 | GEO accession |
| | cancer | | GSE3494: |
| | | | http://www.ncbi.nlm.nih.gov/projects/geo/ |
| Wang | Breast | 286 | GEO accession |
| | cancer | | GSE2034 |
| | | | http://www.ncbi.nlm.nih.gov/projects/geo/ |
| Van de | Breast | 295 | http://microarraypubs.stanford.edu/ |
| Vijver | cancer | | wound_NKI/ |
| Zhao | Renal | 177 | SMD: |
| | cancer | | http://smd.stanford.edu/ |
| Beer | Lung | 86 | http://dot.ped.med.umich.edu:2000/ |
| | cancer | | ourimage/pub/Lung/index.html |

Data Filtering and Pre-Processing

Datasets downloaded from the SMD23 were filtered according to the parameters in the paper. CloneIDs were chosen as gene annotation and the data obtained was log-transformed. For the normalized Affymetrix arrays 24,25 the genes were log-transformed. The Beer et al. (26) dataset was already preprocessed therefore to perform log-transformation all expression values below 1.1 were set to 1.1, this was similar to the processing performed by Chen et al. (2). In all other cases the data was kept in the downloaded format (12), which was already log-transformed. CloneIDs and Affymetrix probeIDs were translated into UnigeneIDs (Build199) with Source (http://smd.stanford.edu/) or Affymetrix data files (Affx annotation files available at www.affymetrix.com). Datasets were imported in Matlab (Matlab 7.1, The Mathworks, Massachusetts, USA). Unless indicated otherwise, analyses are performed in this program.

Signature Score Calculation

Expression data of the genes in the signature was extracted from the dataset. The following step was used to calculate a signature score for each patient in the dataset. This score was defined as the weighted average expression value of the genes in the signature. A weight of −1 or 1 was assigned to each gene, dependent on the phenotype the gene represents (supplementary material). The signature score then reflects the status of the studied process in a tumor. When a gene was represented by more than one probe on an array, the expression of the probes was averaged before signature calculation.

Statistical Analysis

A loop of 1,000 clustering repeats with the K-means clustering function in Matlab was applied to split the patients in two groups according to their signature score. Outcome in the two groups was analyzed and compared by the Kaplan-Meier method. Differences in outcome were tested for statistical significance by the log-rank test for different common end-points. For breast and renal cancer the common end-points are 5-years and 10 years survival, for lung cancer these are 2-years and 5-years survival, all end-points are analyzed when follow-up is long enough. Results for the log-rank tests are given as the average, standard deviation and the range of the p-values, also the percentage of p-values from the 1,000 clustering runs that were significant was calculated to evaluate the prognostic power of the signature and stability of the clustering. Multivariate Cox regression analysis with stepwise backward selection procedure was performed in SPSS (SPSS 12.0.1, SPSS Inc, Illinois, USA) to show the clinical relevance of the proliferation signature. Further Matlab was used to integrate all parameters in a model and evaluate the area under the curve (AUC) of the model with and without addition of the signature to the clinical parameters; details of the methodology are given in the supplementary data.

Random Signature Testing

A method to test a predefined number of random signatures of a predefined size on all the datasets was developed. To show the strength of the best proliferation signature 10,000 random generated gene sets, with sizes equal to the size of the best proliferation signature, were tested on the datasets. These random gene sets were generated and tested in a similar manner as the proliferation signatures.

Results

Comparison of Two Proliferation Signatures

Two proliferation signatures were derived from literature. Signature 1 (Whitfield et al. (22)) and signature 2 (Chang et al. (6)) consist of respectively 1,134 and 199 cloneIDs, these map to 815 and 154 unique UnigeneIDs, respectively. The distribution of genes in the different cell cycle phases for the two signatures is distinct (Supplementary data Table 1), indicating that the signatures are different. Signature 1 shows equal proportions of genes in the defined cell cycle phases. However in signature 2 more genes are involved in G2 and clearly less genes are involved in M/G1. Outcome prediction with proliferation signatures The signatures were tested on several publicly available microarray datasets (Table 2). The signatures were evaluated using a signature score. To calculate the signature score, weights had to be defined for each gene. After translation and weight assignment several genes were discarded from analyses, for these genes weight assignment was ambiguous, details are provided in the supplementary material. The final signatures consist of respectively 508 and 110 UnigeneIDs for signature 1 and 2.

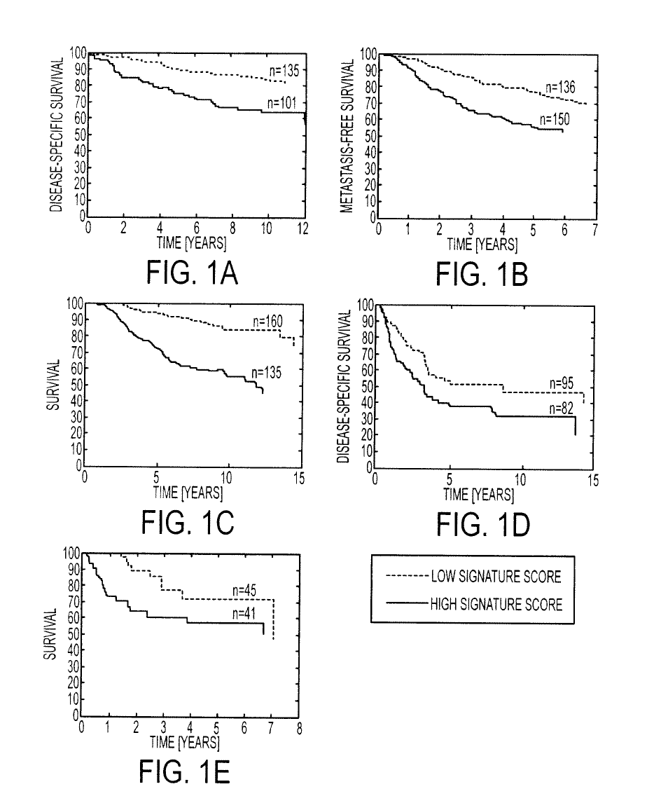

In every dataset a signature score was calculated for each patient. The patients were separated in two groups by clustering these signature scores. Results of the log-rank tests are given in Table 3 and in FIG. 1 the Kaplan-Meier curves for signature 2 are shown. Signature 2 gives clear risk stratification in all datasets, all p-values of the 1000 clustering runs<0.05. Results of the log-rank test show not only that signature 2 gives a better risk stratification than signature 1, also the overall robustness of the separation is stronger, indicated by the small standard deviations. Nevertheless both signatures show very good prognostic value on the three breast cancer datasets. The range and standard deviations of the 1,000 clustering runs also show that the results are stable for these datasets.

TABLE 3
Results of log-rank test for the signatures
for the different end-points

| | | | | | |
|---|---|---|---|---|---|
| | | | | | % of |
| | End- | P- | | | significant |
| Dataset | point | value | SD | Range | runs |

Signature 1

| | | | | | |
|---|---|---|---|---|---|
| Miller | 5-years | 3.1 10⁻³ | 1.6 10⁻³ | 1.5 10⁻³-4.7 10⁻³ | 100 |
| | 10-years | 6.9 10⁻⁴ | 3.4 10⁻⁴ | 3.6 10⁻⁴-1.0 10⁻³ | 100 |
| Wang | 5-years | 1.9 10⁻³ | 1.0 10⁻³ | 9.9 10⁻⁴-3.0 10⁻³ | 100 |
| Van de | 5-years | 4.6 10⁻⁵ | 0.0 | 4.6 10⁻⁵-4.6 10⁻⁵ | 100 |
| Vijver | | | | | |
| | 10-years | 2.5 10⁻⁷ | 0.0 | 2.5 10⁻⁷-2.5 10⁻⁷ | 100 |
| Zhao | 5-years | 0.48 | 8.0 10⁻² | 0.39-0.55 | 0 |
| | 10-years | 0.54 | 3.0 10⁻² | 0.51-0.57 | 0 |
| Beer | 2-years | 0.16 | 5.4 10⁻² | 0.12-0.20 | 0 |
| | 5-years | 0.49 | 9.8 10⁻² | 0.11-0.63 | 0 |

Signature 2

| | | | | | |
|---|---|---|---|---|---|
| Miller | 5-years | 4.1 10⁻³ | 1.8 10⁻³ | 2.0 10⁻³-6.2 10⁻³ | 100 |
| | 10-years | 7.0 10⁻⁴ | 1.8 10⁻⁴ | 4.6 10⁻⁴-9.3 10⁻⁴ | 100 |
| Wang | 5-years | 2.3 10⁻³ | 1.8 10⁻⁴ | 5.7 10⁻⁵-6.4 10⁻⁴ | 100 |
| Van de | 5-years | 1.4 10⁻⁶ | 0.0 | 1.4 10⁻⁶-1.4 10⁻⁶ | 100 |
| Vijver | | | | | |
| | 10-years | 3.0 10⁻⁸ | 1.6 10⁻¹⁰ | 3.0 10⁻⁸-3.0 10⁻⁸ | 100 |
| Zhao | 5-years | 3.1 10⁻² | 1.1 10⁻² | 1.9 10⁻²-4.2 10⁻² | 100 |
| | 10-years | 2.3 10⁻² | 3.2 10⁻³ | 2.0 10⁻²-2.7 10⁻² | 100 |
| Beer | 2-years | 3.3 10⁻³ | 2.2 10⁻⁵ | 3.3 10⁻³-3.4 10⁻³ | 100 |
| | 5-years | 2.8 10⁻² | 6.8 10⁻⁵ | 2.8 10⁻²-2.8 10⁻² | 100 |

Statistical Analysis of Signature Scores

Multivariate Cox-regression analyses were performed to investigate whether the association between the best proliferation signature and outcome was independent of clinical prognostic factors. The variables analyzed differed per dataset, since different clinical factors are provided (Supplementary data Table 2). A stepwise backward selection procedure is performed to select the variables that are prognostic factors; the end-point is 10-years for breast and renal cancer and 5-years for lung cancer. Follow-up time in the Wang et al. (25) dataset is not long enough, in that dataset 5-years was used. In Table 4 the factors selected with this procedure are given for all the datasets, choosing another end-point did not influence the results dramatically. In 3 out of 5 datasets the proliferation signature is a significant prognostic factor of outcome.

TABLE 4
Clinical parameters selected with
stepwise backward selection in multivariate
Cox regression analyses including signature 2

| | Hazard ratio | |
|---|---|---|
| | (95% CI) | p-value |
| Miller | | |
| Tumor size | 3.3 (1.7-6.6) | 0.001 |
| LNS\hat{ } | 2.8 (1.6-5.0) | <0.001 |
| Proliferation | 3.4 (1.4-8.2) | 0.005 |
| Wang | | |
| Proliferation | 2.6 (1.5-4.4) | 0.002 |
| Van de Vijver | | |
| Age | 0.95 (0.91-0.99) | 0.027 |
| Tumor size* | 1.5 (0.93-2.5) | 0.096 |
| Elston grade | 2.2 (1.4-3.4) | <0.001 |
| Proliferation | 21 (1.8-234) | 0.015 |
| Zhao | | |
| Performance | 1.3 (1.1-1.6) | 0.007 |
| status | | |
| Grade | 1.5 (1.0-2.1) | 0.026 |
| Stage | 3.3 (2.5-4.4) | <0.001 |
| Beer | | |
| Age | 1.0 (1.0-1.1) | 0.058 |
| Stage | 2.4 (1.6-3.7) | <0.001 |
| Differentiation | 2.0 (1.0-4.0) | 0.046 |

*Categories: ≦2 cm or >2 cm
\hat{ }LNS: lymph-node status

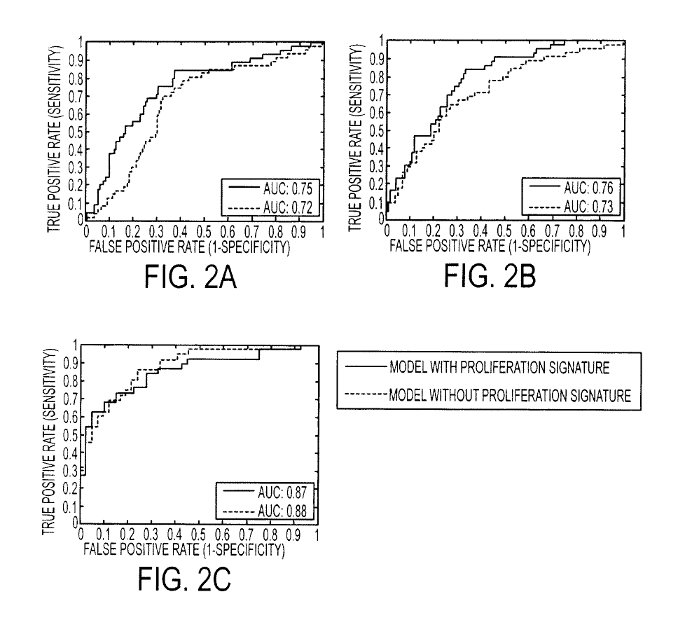

AUCs were calculated for all clinical parameters and the best proliferation signature. Results of this analyses show that the proliferation signature has a high AUC in all datasets (Supplementary data Table 3). To quantify the gain obtained with this signature a model of the clinical factors with and without the signature was generated and evaluated with the AUC (Supplementary data). Only the datasets with more than one clinical parameter and more than 150 patients are included. In two out of three datasets the AUC increased when the proliferation signature was added to the model (FIG. 2). In order to show the strength of the signature, 10,000 random generated signatures were tested on all datasets. Of these 10,000 no signature gave a significant result on all datasets.

Discussion

Application of the signature score methodology used here provides a very stringent method to evaluate the prognostic power of a signature. Typically signature evaluations are conducted by clustering of patients and genes, which can result in clear differences in survival even when gene expression differences are not very large. The employment of a more strict method, like the signature score used here, gives a better indication on the magnitude of association and thus clinical feasibility of the signature. The proliferation signature could be further optimized by weighting genes according to their importance, which can lead to a reduction in signature size. Here equal weights were chosen for all genes even though some may clearly have a more profound role than others. It is likely that this is dependent on the tumor type, since proliferation is one of the pathways almost always disrupted in cancer. In this light signature 2 could be considered as a weighting of signature 1. Several genes do not contribute to prognosis and are therefore assigned a weight of 0.

Many other signatures identified in previous studies include large clusters of proliferation genes (4, 9, 17-20, 27-29). Some even refer to their signature as a proliferation signature (4, 29). However in these supervised studies not all genes in the signature are related to proliferation and can therefore not be referred to strictly as general proliferation signatures. Dai et al. (4) determined a supervised signature which was associated with metastasis. Many of the identified genes were related to the cell cycle and these authors thus referred to their classifier as a proliferation signature. However only 17 out of 50 genes in this signature are cell cycle related when compared to the initial gene list of Whitfield et al. (22). Further the experimental method was not designed to find a proliferation signature. The same applies to the study of Rosenwald et al. (29), only 28 of the 48 genes that were associated with length of survival are related to proliferation.

A proliferation signature was derived from in vitro microarray studies based only on genes that differ in expression in different parts of the cell cycle (6, 22). Results show that the proliferation signature has a high value in patient risk stratification in several types of cancer and can be combined with other phenotype based signatures, like the IGS. Combining the proliferation and wound signature will not increase the prognostic power, as they primarily identify the same patients. This and the fact that large clusters of proliferation genes are identified in many gene signatures (4, 9, 17-20, 27, 28) raise the possibility that many of these signatures, including the wound signature, might be driven by proliferation. Fan et al. (11) already suggested that many signatures probably track a common set of biologic phenotypes and have therefore a similar prognostic strength. The proliferation signature has a high prognostic power, like many signatures, however it is one of the few signatures that has a potential predictive value. It can possibly be used to prescribe a treatment targeting tumor proliferation. Studies indicate that fast proliferating tumors can benefit from accelerated radiotherapy or chemo-radiotherapy (30, 31). The proliferation signature could be used as a predictive test for patient selection for these treatments. This should be tested in randomized patient trials.

Previous studies have tried to assess the predictive value of proliferation by means of Ki67 staining, measurement of labeling index (LI) and potential doubling time (Tpot) calculation. Overall results of these single-parameter indicators are disappointing, however in several studies a weak prediction potential is found (30, 32, 33). This can be due to the large chance of misclassification with these single-parameter indicators (16, 34). Application of multi-parameter indicators, like the proliferation signature, is therefore a more attractive method (16). In conclusion, the application of phenotype based signatures like the proliferation signature can be used in patient risk stratification, in addition to clinical parameters. It has a high prognostic value and unlike other signatures it has the potential to be converted into a predictive test. It is proposed that patients with a high proliferation signature score could benefit from accelerated radiotherapy or chemo-radiotherapy.

### Supplementary Materials and Methods

Signature Processing

For all signatures the gene identifiers were translated into UnigeneIDs (Build199) with Source (http://smd.stanford.edu/) or Affymetrix data files (Affx annotation files (www.affymetrix.com)). After this translation several genes in the proliferation signatures were represented by more than one CloneID. In case these cloneIDs represented the same proliferation status they were included in the signature. However when multiple cloneIDs representing one gene corresponded to different proliferation conditions these genes were discarded. This was approximately 3% of the genes in each signature.

Data Filtering and Pre-Processing

Datasets downloaded from the SMD (23) were filtered according to the parameters in the paper. CloneIDs were chosen as gene annotation and the data obtained was log-transformed. For the normalized Affymetrix arrays (24, 25) the genes were log-transformed. The Beer et al. (26) dataset was already preprocessed therefore to perform log-transformation all expression values below 1.1 were set to 1.1, this was similar to the processing performed by Chen et al. (2). In all other cases the data was kept in the downloaded format (12), which was already log-transformed. CloneIDs and Affymetrix probeIDs were translated into UnigeneIDs (Build199).

Weight Assignment

The weights for the genes in the proliferation signatures were defined as −1 when a gene represents non-proliferating cells (G₁ phase) and 1 if a gene represents proliferating cells (S, G₂ and M phase). The given cell cycle phases are G₁/S, S, G₂, G₂/M and M/G₁ (Supplementary data Table 1). It is clear that the genes with a peak expression in the phases S, G₂ and G₂/M were assigned a weight of 1. However it was unclear what weight should be assigned to the genes in G₁/S and M/G₁. Therefore these genes, 34% and 25% of the genes in signature 1 and 2 respectively, were omitted from further analyses. The final signatures consisted respectively of 508 and 110 unique UnigeneIDs (build #199) for signature 1 and 2. The gene lists for signature 2 are provided in Table 1 above.

AUC Model Calculation

Matlab (Matlab 7.1, The Mathworks, Massachusetts, USA) was used to integrate all parameters in a model and evaluate the area under the curve (AUC) of the model with and without addition of the signature to the clinical parameters. All clinical parameters were transformed to numbers, to be able to incorporate them in Matlab, e.g. negative and positive ER-status were set to 0 and 1 respectively. These parameters were incorporated in a model with the classify function of Matlab, which used the diaglinear method. Part of the dataset was used as training set and the other part as a test set. Assignment of samples to test and training set was done at random and repeated 1,000 times.

Contingency Table Analyses

Contingency tables were used to compare patient classification of the proliferation signature to the patient classification of other gene signatures. For three datasets (12, 24, 26) the group classification of the gene signatures were identified in these studies: the 32-gene p53 signature (24), the 70-gene signature (12) and the 100 survival related genes (26). These and the wound response and IGS signature were evaluated.

Contingency tables were evaluated with the p-value calculated from Chi-square test and the Cramer's V statistic. The Cramer's V statistic (value can range from 0 to 1) measures the strength of association between the two variables analyzed in the contingency table, with 1 indicating perfect association and 0 indicating no association. Values between 0.36 and 0.49 indicate a substantial relation between the signatures and values>0.50 indicate a strong relation (11).

Supplementary Tables

SUPPLEMENTARY TABLE 1
Percentage of genes in the different cell cycle
phases in the two proliferation signatures
(numbers are given between brackets)

| Cell cycle phase | Signature 1 | Signature 2 |
|---|---|---|
| G₁/S | 18.0 (147) | 18.8 (29) |
| S | 18.0 (147) | 19.5 (30) |
| G₂ | 19.5 (159) | 26.0 (40) |
| G₂/M | 21.7 (177) | 22.1 (34) |
| M/G₁ | 16.0 (130) | 6.5 (10) |
| Matching combinations* | 3.1 (25) | 3.9 (6) |
| Non-matching combinations^{†} | 3.7 (30) | 3.3 (5) |
| G₁/S | 18.0 (147) | 18.8 (29) |
| S | 18.0 (147) | 19.5 (30) |
| G₂ | 19.5 (159) | 26.0 (40) |
| G₂/M | 21.7 (177) | 22.1 (34) |
| M/G₁ | 16.0 (130) | 6.5 (10) |
| Matching combinations* | 3.1 (25) | 3.9 (6) |
| Non-matching combinations^{†} | 3.7 (30) | 3.3 (5) |

*Different cloneIDs for 1 UnigeneID are found in different phases, but all phases represent the same proliferation status (i.e S, G₂, G₂/M)
^{†}Different cloneIDs for 1 UnigeneID are found in different phases and the phases represent a different proliferation status (i.e G₂/M, G₁/S)

SUPPLEMENTARY TABLE 2
Results of log-rank test for the signatures for the
different end-points

| | | | | | |
|---|---|---|---|---|---|
| Dataset | End-point | P-value | SD | Range | % of significant runs |

Signature 1

| | | | | | |
|---|---|---|---|---|---|
| Miller | 5-years | 3.1 10⁻³ | 1.6 10⁻³ | 1.5 10⁻³-4.7 10⁻³ | 100 |
| | 10-years | 6.9 10⁻⁴ | 3.4 10⁻⁴ | 3.6 10⁻⁴-1.0 10⁻³ | 100 |
| Wang | 5-years | 1.9 10⁻³ | 1.0 10⁻³ | 9.9 10⁻⁴-3.0 10⁻³ | 100 |
| Van de Vijver | 5-years | 4.6 10⁻⁵ | 0.0 | 4.6 10⁻⁵-4.6 10⁻⁵ | 100 |
| | 10-years | 2.5 10⁻⁷ | 0.0 | 2.5 10⁻⁷-2.5 10⁻⁷ | 100 |
| Zhao | 5-years | 0.48 | 8.0 10⁻² | 0.39-0.55 | 0 |
| | 10-years | 0.54 | 3.0 10⁻² | 0.51-0.57 | 0 |
| Beer | 2-years | 0.16 | 5.4 10⁻² | 0.12-0.20 | 0 |
| | 5-years | 0.49 | 9.8 10⁻² | 0.11-0.63 | 0 |

Signature 2

| | | | | | |
|---|---|---|---|---|---|
| Miller | 5-years | 4.1 10⁻³ | 1.8 10⁻³ | 2.0 10⁻³-6.2 10⁻³ | 100 |
| | 10-years | 7.0 10⁻⁴ | 1.8 10⁻⁴ | 4.6 10⁻⁴-9.3 10⁻⁴ | 100 |
| Wang | 5-years | 2.3 10⁻³ | 1.8 10⁻⁴ | 5.7 10⁻⁵-6.4 10⁻⁴ | 100 |
| Van de Vijver | 5-years | 1.4 10⁻⁶ | 0.0 | 1.4 10⁻⁶-1.4 10⁻⁶ | 100 |
| | 10-years | 3.0 10⁻⁸ | 1.6 10⁻¹⁰ | 3.0 10⁻⁸-3.0 10⁻⁸ | 100 |
| Zhao | 5-years | 3.1 10⁻² | 1.1 10⁻² | 1.9 10⁻²-4.2 10⁻² | 100 |
| | 10-years | 2.3 10⁻² | 3.2 10⁻³ | 2.0 10⁻²-2.7 10⁻² | 100 |
| Beer | 2-years | 3.3 10⁻³ | 2.2 10⁻⁵ | 3.3 10⁻³-3.4 10⁻³ | 100 |
| | 5-years | 2.8 10⁻² | 6.8 10⁻⁵ | 2.8 10⁻²-2.8 10⁻² | 100 |

SUPPLEMENTARY TABLE 3
AUCs of individual clinical parameters and proliferation signature 2

| Miller | | Wang | | Van de Vijver | | Zhao | | Beer | |
|---|---|---|---|---|---|---|---|---|---|
| Age | 0.48 | ER-status | 0.62 | Age | 0.42 | Age | 0.43 | Age | 0.60 |
| Elston grade | 0.65 | Proliferation** | 0.59 | Elston grade | 0.72 | Sex | 0.45 | Sex | 0.45 |
| Tumor size* | 0.71 | | | Tumor size* | 0.67 | Performance status | 0.60 | Smoking^{†} | 0.49 |
| ER-status | 0.58 | | | ER-status | 0.41 | Grade | 0.64 | Stage | 0.66 |
| LNS^{‡} | 0.70 | | | LNS^{‡} | 0.52 | Stage | 0.85 | Differentiation | 0.59 |
| PgR^{§} | 0.54 | | | NIH risk | 0.66 | Proliferation** | 0.56 | K-ras mutation | 0.52 |
| P53-status | 0.57 | | | Mastectomy | 0.57 | | | Proliferation** | 0.64 |
| Proliferation** | 0.67 | | | Chemotherapy | 0.52 | | | | |
| | | | | Hormone therapy | 0.53 | | | | |
| | | | | Proliferation** | 0.72 | | | | |

*Categories: ≦2 cm or >2 cm
^{†}Categories: smoker or non-smoker
^{‡}LNS: lymph-node status
^{§}PgR: progesterone receptor status
**proliferation: proliferation signature 2

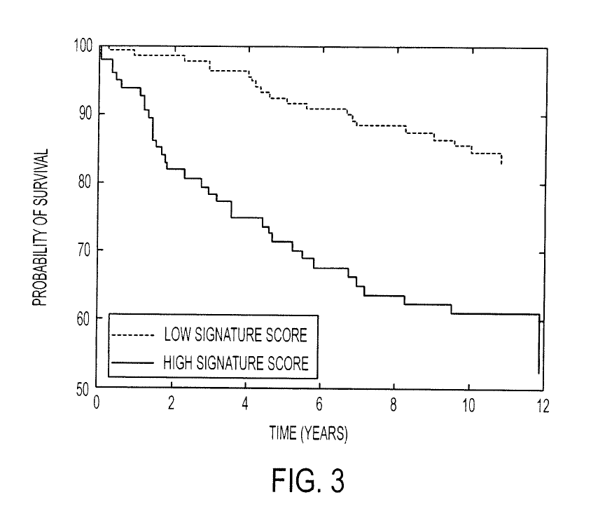

In further experiments the gene signature of (a) was further reduced to provide gene signatures (b)-(k). A signature score was calculated for each patient in the different datasets using each signature. These scores were used to cluster the patients in two groups, one with low expression and one with high expression of the signature. Kaplan-Meier survival curves for the two groups were compared in FIGS. 3-12. Patients with tumors with a high proliferation signature score had worse outcomes than those with tumors with a low proliferation signature score.

Gene signature (a) was further reduced to gene signature (b) which is 5 genes represented by Unigene ID Nos.: Hs.524571, Hs.226390, Hs.436187, Hs.472716, and Hs.194698. When run on the Miller data set (with one round AUC criteria) Kaplan Meier curves were produced in FIG. 3 (AUC: 0.6943, Plogrank: 0.0000, Pcox: 0.0000, CI: 0.6172).

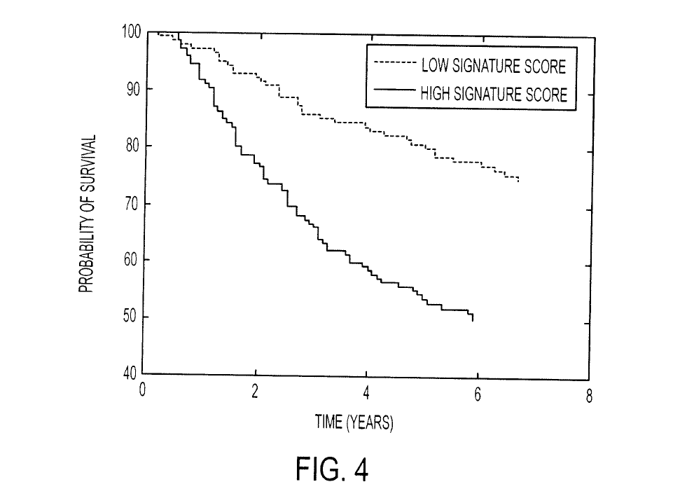

Gene signature (a) was further reduced to gene signature (c) which is 5 genes represented by Unigene ID Nos.: Hs.368563, Hs.444028, Hs.58992, Hs.575032, and Hs.591697. When run on the Wang data set (with one round AUC criteria) Kaplan Meier curves were produced in FIG. 4 (AUC: 0.6900, Plogrank: 0.0000, Pcox: 0.0000, CI: 0.6170).

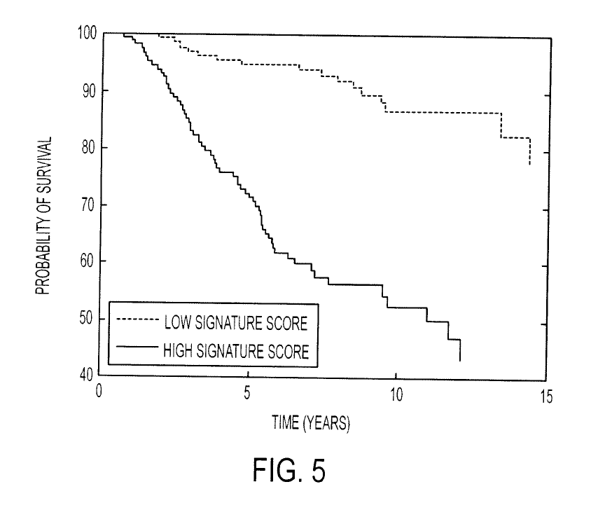

Gene signature (a) was further reduced to gene signature (d) which is 5 genes represented by Unigene ID Nos.: Hs.436187, Hs.194698, Hs.250822, Hs.93002, and Hs.308045. When run on the van de Vijver data set (with one round AUC criteria) Kaplan Meier curves were produced in FIG. 5 (AUC: 0.7576, Plogrank: 0.0000, Pcox: 0.0000, CI: 0.5694).

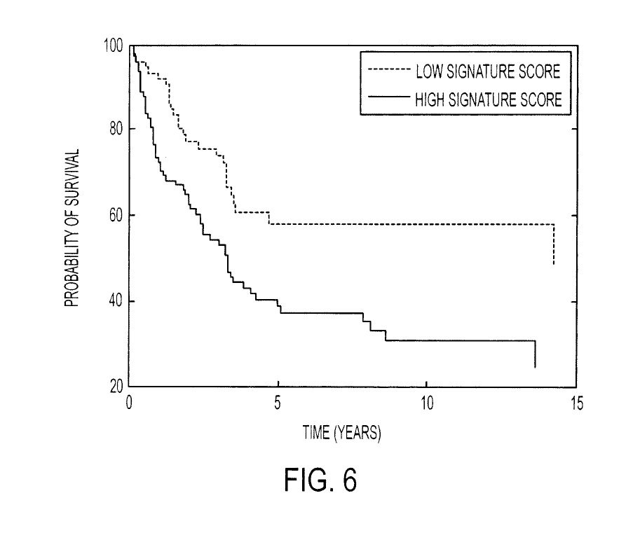

Gene signature (a) was further reduced to gene signature (e) which is 5 genes represented by Unigene ID Nos.: Hs.58992, Hs.522632, Hs.446017, Hs.240, and Hs.533059. When run on the Zhao data set (with one round AUC criteria) Kaplan Meier curves were produced in FIG. 6 (AUC: 0.6438, Plogrank: 0.0021, Pcox: 0.0030, CI: 0.4925).

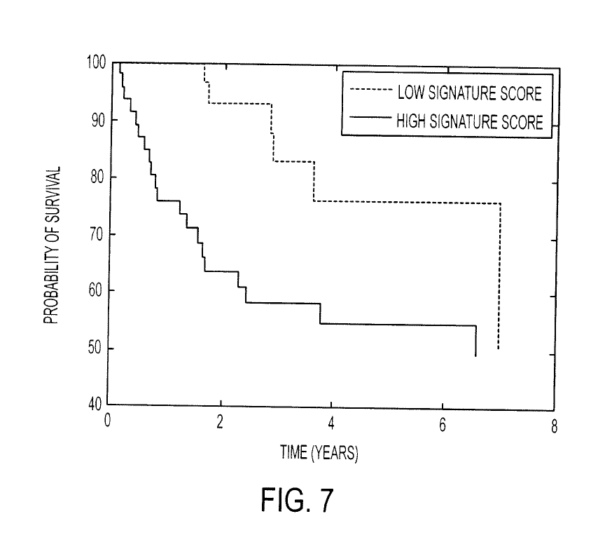

Gene signature (a) was further reduced to gene signature (f) which is 5 genes represented by Unigene ID Nos.: Hs.58974, Hs.75318, Hs.506652, H5.184339, and Hs.81892. When run on the Beer data set (with one round AUC criteria) Kaplan Meier curves were produced in FIG. 7 (AUC: 0.6865, Plogrank: 0.0165, Pcox: 0.0152, CI: 0.4776).

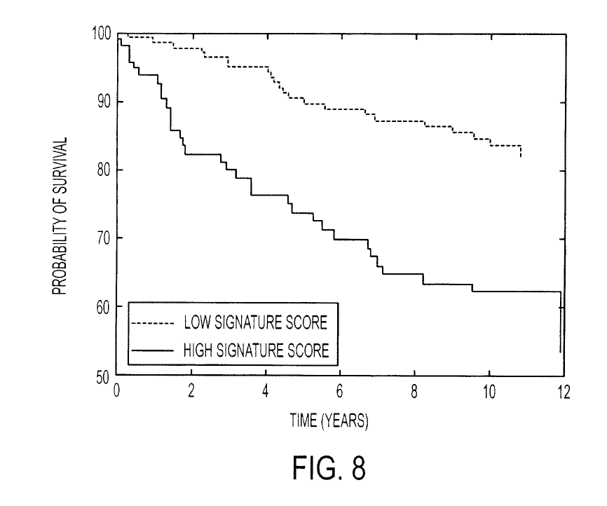

Gene signature (a) was further reduced to gene signature (g) which is 10 genes represented by Unigene ID Nos.: Hs.524571, Hs.226390, Hs.436187, Hs.472716, Hs.194698, Hs.386189, Hs.409065, Hs.5199, Hs.434250, and Hs.93002. When run on the Miller data set (with one round AUC criteria) Kaplan Meier curves were produced in FIG. 8 (AUC: 0.6911, Plogrank: 0.0001, Pcox: 0.0000, CI: 0.6272).

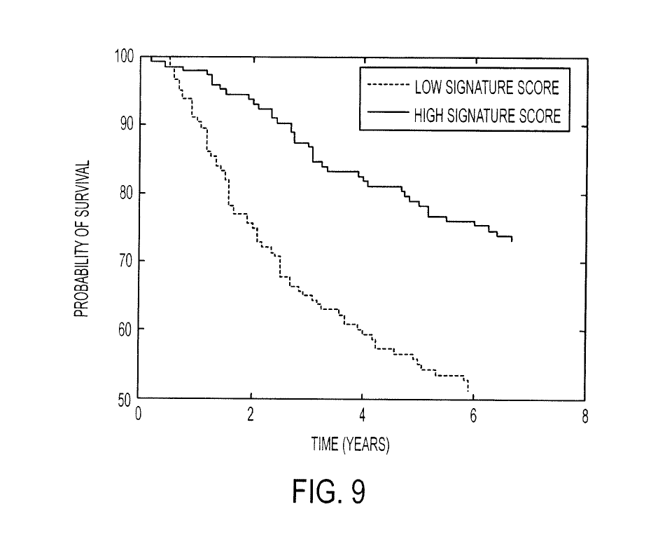

Gene signature (a) was further reduced to gene signature (h) which is 10 genes represented by Unigene ID Nos.: SEQ ID NOS: 88, 45, 31, 102, 32, 75, 57, 79, 51, and 74. When run on the Wang data set (with one round AUC criteria) Kaplan Meier curves were produced in FIG. 9 (AUC: 0.6684, Plogrank: 0.0000, Pcox: 0.0000, CI: 0.6182).

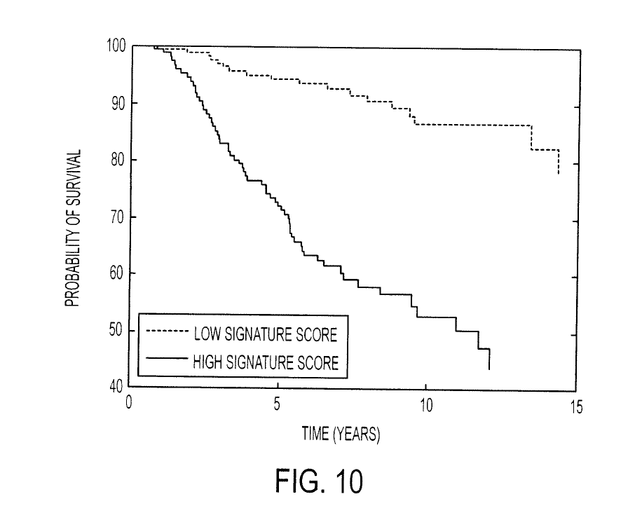

Gene signature (a) was further reduced to gene signature (i) which is 10 genes represented by Unigene ID Nos.: Hs.436187, Hs.194698, Hs.250822, Hs.93002, Hs.308045, Hs.444082, Hs.1594, Hs.184339, Hs.5199, Hs.409065. When run on the van de Vijver data set (with one round AUC criteria) Kaplan Meier curves were produced in FIG. 10 (AUC: 0.7551, Plogrank: 0.0000, Pcox: 0.0000, CI: 0.5710).

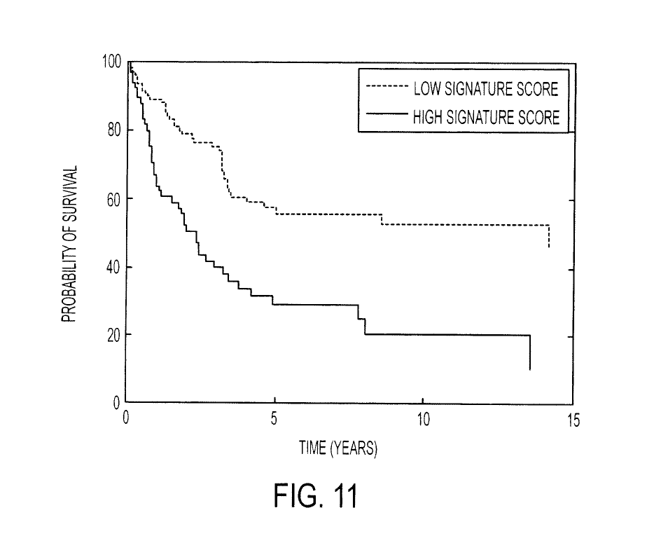

Gene signature (a) was further reduced to gene signature (j) which is 10 genes represented by Unigene ID Nos.: Hs.58992, Hs.522632, Hs.446017, Hs.240, Hs.533059, Hs.513126, Hs.132966, Hs.532803, Hs.239, and Hs.58974. When run on the Zhao data set (with one round AUC criteria) Kaplan Meier curves were produced in FIG. 11 (AUC: 0.6531, Plogrank: 0.0000, Pcox: 0.0003, CI: 0.5050).

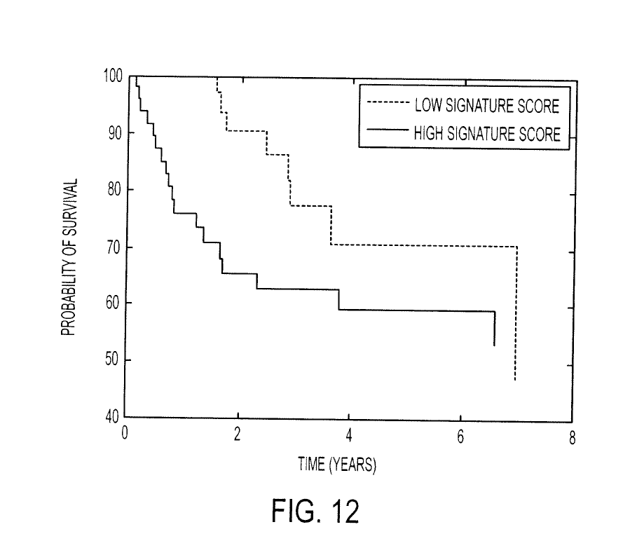

Gene signature (a) was further reduced to gene signature (k) which is 10 genes represented by Unigene ID Nos.: Hs.58974, Hs.75318, Hs.506652, Hs.184339, Hs.81892, Hs.591322, Hs.156346, Hs.72550, Hs.374378, and Hs.77695. When run on the Beer data set (with one round AUC criteria) Kaplan Meier curves were produced in FIG. 12 (AUC: 0.6840, Plogrank: 0.0911, Pcox: 0.0125, CI: 0.4881).

### REFERENCES

1. Bild A H, Potti A, Nevins J R. Linking oncogenic pathways with therapeutic opportunities. Nat Rev Cancer 2006; 6(9):735-41.

2. Chen H Y, Yu S L, Chen C H, et al. A five-gene signature and clinical outcome in non-small-cell lung cancer. N Engl J Med 2007; 356(1):11-20.

3. Quackenbush J. Microarray analysis and tumor classification. N Engl J Med 2006; 354(23):2463-72.

4. Dai H, van't Veer L, Lamb J, et al. A cell proliferation signature is a marker of extremely poor outcome in a subpopulation of breast cancer patients. Cancer Res 2005; 65(10):4059-66.

5. Dupuy A, Simon R M. Critical review of published microarray studies for cancer outcome and guidelines on statistical analysis and reporting. J Natl Cancer Inst 2007; 99(2):147-57.

6. Chang H Y, Sneddon J B, Alizadeh A A, et al. Gene expression signature of fibroblast serum response predicts human cancer progression: similarities between tumors and wounds. PLoS Biol 2004; 2(2):E7.

7. Chi J T, Wang Z, Nuyten D S, et al. Gene expression programs in response to hypoxia: cell type specificity and prognostic significance in human cancers. PLoS Med 2006; 3(3):e47.

8. Sung F L, Hui EP, Tao Q, et al. Genome-wide expression analysis using microarray identified complex signaling pathways modulated by hypoxia in nasopharyngeal carcinoma. Cancer Lett 2007.

9. Liu R, Wang X, Chen G Y, et al. The prognostic role of a gene signature from tumorigenic breast-cancer cells. N Engl J Med 2007; 356(3):217-26.

10. Chang H Y, Nuyten D S, Sneddon J B, et al. Robustness, scalability, and integration of a wound-response gene expression signature in predicting breast cancer survival. Proc Natl Acad. Sci USA 2005; 102(10):3738-43.

11. Fan C, Oh D S, Wessels L, et al. Concordance among gene-expression-based predictors for breast cancer. N Engl J Med 2006; 355(6):560-9.

12. van de Vijver M J, He Y D, van't Veer L J, et al. A gene-expression signature as a predictor of survival in breast cancer. N Engl J Med 2002; 347(25):1999-2009.

13. Adler A S, Lin M, Horlings H, Nuyten D S, van de Vijver M J, Chang H Y. Genetic regulators of large-scale transcriptional signatures in cancer. Nat Genet 2006; 38(4):421-30.

14. Bourhis J, Overgaard J, Audry H, et al. Hyperfractionated or accelerated radiotherapy in head and neck cancer: a meta-analysis. Lancet 2006; 368(9538):843-54.

15. De Ruysscher D, Pijls-Johannesma M, Bentzen S M, et al. Time between the first day of chemotherapy and the last day of chest radiation is the most important predictor of survival in limited-disease small-cell lung cancer. J Clin Oncol 2006; 24(7):1057-63.

16. Whitfield M L, George L K, Grant G D, Perou C M. Common markers of proliferation. Nat. Rev Cancer 2006; 6(2):99-106.

17. Perou C M, Jeffrey S S, van de Rijn M, et al. Distinctive gene expression patterns in human mammary epithelial cells and breast cancers. Proc Natl Acad Sci USA 1999; 96(16):9212-7.

18. Welsh J B, Zarrinkar P P, Sapinoso L M, et al. Analysis of gene expression profiles in normal and neoplastic ovarian tissue samples identifies candidate molecular markers of epithelial ovarian cancer. Proc Natl Acad Sci USA 2001; 98(3):1176-81.

19. Pawitan Y, Bjohle J, Amler L, et al. Gene expression profiling spares early breast cancer patients from adjuvant therapy: derived and validated in two population-based cohorts. Breast Cancer Res 2005; 7(6):R953-64.

20. Sotiriou C, Wirapati P, Loi S, et al. Gene expression profiling in breast cancer: understanding the molecular basis of histologic grade to improve prognosis. J Natl Cancer Inst 2006; 98(4):262-72.

21. Paik S, Shak S, Tang G, et al. A multigene assay to predict recurrence of tamoxifentreated, node-negative breast cancer. N Engl J Med 2004; 351(27):2817-26.

22. Whitfield M L, Sherlock G, Saldanha A J, et al. Identification of genes periodically expressed in the human cell cycle and their expression in tumors. Mol Biol Cell 2002; 13(6):1977-2000.

23. Zhao H, Ljungberg B, Grankvist K, Rasmuson T, Tibshirani R, Brooks J D. Gene expression profiling predicts survival in conventional renal cell carcinoma. PLoS Med 2006; 3(1):e13.

24. Miller L D, Smeds J, George J, et al. An expression signature for p53 status in human breast cancer predicts mutation status, transcriptional effects, and patient survival. Proc Natl. Aad Sci USA 2005; 102(38):13550-5.

25. Wang Y, Klijn J G, Zhang Y, et al. Gene-expression profiles to predict distant metastasis of lymph-node-negative primary breast cancer. Lancet 2005; 365(9460):671-9.

26. Beer D G, Kardia S L, Huang C C, et al. Gene-expression profiles predict survival of patients with lung adenocarcinoma. Nat Med 2002; 8(8):816-24.

27. Ross D T, Scherf U, Eisen M B, et al. Systematic variation in gene expression patterns in human cancer cell lines. Nat Genet 2000; 24(3):227-35.

28. van't Veer L J, Dai H, van de Vijver M J, et al. Gene expression profiling predicts clinical outcome of breast cancer. Nature 2002; 415(6871):530-6.

29. Rosenwald A, Wright G, Wiestner A, et al. The proliferation gene expression signature is a quantitative integrator of oncogenic events that predicts survival in mantle cell lymphoma. Cancer Cell 2003; 3(2):185-97.

30. Gasinska A, Fowler J F, Lind B K, Urbanski K. Influence of overall treatment time and radiobiological parameters on biologically effective doses in cervical cancer patients treated with radiation therapy alone. Acta Oncol 2004; 43(7):657-66.

31. Corvo R, Paoli G, Giaretti W, et al. Evidence of cell kinetics as predictive factor of response to radiotherapy alone or chemoradiotherapy in patients with advanced head and neck cancer. Int J Radiat Oncol Biol Phys 2000; 47(1):57-63.

32. Begg A C, Haustermans K, Hart A A, et al. The value of pretreatment cell kinetic parameters as predictors for radiotherapy outcome in head and neck cancer: a multicenter analysis. Radiother Oncol 1999; 50(1):13-23.

33. Struikmans H, Kal H B, Hordijk G J, van der Tweel I. Proliferative capacity in head and neck cancer. Head Neck 2001; 23(6):484-91.

34. Jalava P, Kuopio T, Juntti-Patinen L, Kotkansalo T, Kronqvist P, Collan Y. Ki67 immunohistochemistry: a valuable marker in prognostication but with a risk of misclassification: proliferation subgroups formed based on Ki67 immunoreactivity and standardized mitotic index. Histopathology 2006; 48(6):674-82.

As a person skilled in the art will readily appreciate, the above description is meant as an illustration of implementation of the principles this invention. This description is not intended to limit the scope or application of this invention in that the invention is susceptible to modification, variation and change, without departing from spirit of this invention, as defined in the following claims.

What is claimed:

1. A method for predicting patient response to cancer treatment, comprising:
measuring in a biological sample from a patient the levels of gene expression of a plurality of genes selected from the groups consisting of Group A, B, C, D, E, F, G, H, I, J, and K, defined below:
a. Group A: Genes corresponding to transcripts associated with the Unigene ID Nos. Hs.121025, Hs.126714, Hs.132966, Hs.141125, Hs.156346, Hs.184339, Hs.1973, Hs.270845, Hs.294088, Hs.300701, Hs.308045, Hs.334562, Hs.339665, Hs.369279, Hs.405925, Hs.418533, Hs.433615, Hs.434250, Hs.435570, Hs.436912, Hs.438550, Hs.446017, Hs.472716, Hs.477879, Hs.503749, Hs.522632, Hs.524571, Hs.532968, Hs.533059, Hs.535012, Hs.58992, Hs.591697, Hs.603315, Hs.613351, Hs.642598, Hs.656, Hs.75318, Hs.88523, Hs.89497, Hs.93002, Hs.615092, Hs.62180, Hs.532803, Hs.240, Hs.444028, Hs.58974, Hs.104019, Hs.1594, Hs.178695, Hs.183800, Hs.194698, Hs.20575, Hs.226755, Hs.234545, Hs.239, Hs.244580, Hs.250822, Hs.28465, Hs.368710, Hs.374378, Hs.386189, Hs.436187, Hs.469649, Hs.476306, Hs.482233, Hs.497741, Hs.506652, Hs.509008, Hs.514033, Hs.514527, Hs.592049, Hs.592116, Hs.593658, Hs.631699, Hs.631750, Hs.644048, Hs.72550, Hs.75066, Hs.77695, Hs.83758, Hs.152385, Hs.165607, Hs.203965, Hs.208912, Hs.226390, Hs.26516, Hs.35086, Hs.368563, Hs.403171, Hs.409065, Hs.434886, Hs.436341, Hs.444082, Hs.485640, Hs.498248, Hs.513126, Hs.5199, Hs.520943, Hs.534339, Hs.558393, Hs.567267, Hs.575032, Hs.591046, Hs.591322, Hs.592338, Hs.81892, Hs.83765, Hs.88663, Hs.99480, and Hs.484950;
b. Group B: Genes corresponding to transcripts associated with the Unigene ID Nos. Hs.524571, Hs.226390, Hs.436187, Hs.472716, and Hs.194698;
c. Group C: Genes corresponding to transcripts associated with the Unigene ID Nos. Hs.368563, Hs.444028, Hs.58992, Hs.575032, and Hs.591697;
d. Group D: Genes corresponding to transcripts associated with the Unigene ID Nos. Hs.436187, Hs.194698, Hs.250822, Hs.93002, and Hs.308045;
e. Group E: Genes corresponding to transcripts associated with the Unigene ID Nos. Hs.58992, Hs.522632, Hs.446017, Hs.240, and Hs.533059;
f. Group F: Genes corresponding to transcripts associated with the Unigene ID Nos. Hs.58974, Hs.75318, Hs.506652, Hs.184339, and Hs.81892;
g. Group G: Genes corresponding to transcripts associated with the Unigene ID Nos. Hs.524571, Hs.226390, Hs.436187, Hs.472716, Hs.194698, Hs.386189, Hs.409065, Hs.5199, Hs.434250, and Hs.93002;
h. Group H: Genes corresponding to transcripts associated with the Unigene ID Nos. Hs.368563, Hs.444028, Hs.58992, Hs.575032, Hs.591697, Hs.631750, Hs.250822, Hs.77695, Hs.194698, and Hs.631699;
i. Group I: Genes corresponding to transcripts associated with the Unigene ID Nos. Hs.436187, Hs.194698, Hs.250822, Hs.93002, Hs.308045, Hs.444082, Hs.1594, Hs.184339, Hs.5199, and Hs.409065;
j. Group J: Genes corresponding to transcripts associated with the Unigene ID Nos. Hs.58992, Hs.522632, Hs.446017, Hs.240, Hs.533059, Hs.513126, Hs.132966, Hs.532803, Hs.239, and Hs.58974; and
k. Group K: Genes corresponding to transcripts associated with the Unigene ID Nos. Hs.58974, Hs.75318, Hs.506652, Hs.184339, Hs.81892, Hs.591322, Hs.156346, Hs.72550, Hs.374378, and Hs.77695;
creating a signature score from said levels of gene expression; and
correlating the signature score with a predicted response to cancer treatment.

2. The method of claim 1, wherein the levels of gene expression are measured by determining the levels of expression of a group of polynucleotide sequences selected from the group consisting of:
l. the sequences SEQ ID NOS: 1-110;
m. the sequences SEQ ID NOS: 27, 85, 62, 23, and 51;
n. the sequences SEQ ID NOS: 88, 45, 31, 102, and 32;
o. the sequences SEQ ID NOS: 62, 51, 57, 40, and 11;
p. the sequences SEQ ID NOS: 31, 26, 22, 44, and 29;
q. the sequences SEQ ID NOS: 46, 37, 67, 6, and 106;
r. the sequences SEQ ID NOS: 27, 85, 62, 23, 51, 61, 90, 97, 18, and 40;
s. the sequences SEQ ID NOS: 88, 45, 31, 102, 32, 75, 57, 79, 51, and 74;
t. the sequences SEQ ID NOS: 62, 51, 57, 40, 11, 93, 48, 6, 97, and 90;
u. the sequences SEQ ID NOS: 31, 26, 22, 44, 29, 96, 3, 43, 55, and 46; and
v. the sequences SEQ ID NOS: 46, 37, 67, 6, 106, 104, 5, 77, 60, and 79.

3. The method of claim 1, wherein said cancer is breast, renal, or lung cancer.

4. The method of claim 3, wherein said measuring is carried out on RNA from said biological sample.

5. The method of claim 4, wherein said biological sample is from a tumor, a cancerous tissue, a pre-cancerous tissue, a biopsy, a tissue, lymph node, a surgical excision, blood, serum, urine, an organ, or saliva.

6. The method of claim 1, wherein the cancer treatment comprises radiotherapy, fractionated radiotherapy, chemotherapy, or chemo-radiotherapy.

7. A microarray comprising:
a solid substrate and a plurality of nucleic acid probes capable of detecting the levels of gene expression of a plurality of genes selected from the groups consisting of Group A, B, C, D, E, F, G, H, I, J, and K, defined below:
a. Group A: Genes corresponding to transcripts associated with the Unigene ID Nos. Hs.121025, Hs.126714, Hs.132966, Hs.141125, Hs.156346, Hs.184339, Hs.1973, Hs.270845, Hs.294088, Hs.300701, Hs.308045, Hs.334562, Hs.339665, Hs.369279, Hs.405925, Hs.418533, Hs.433615, Hs.434250, Hs.435570, Hs.436912, Hs.438550, Hs.446017, Hs.472716, Hs.477879, Hs.503749, Hs.522632, Hs.524571, Hs.532968, Hs.533059, Hs.535012, Hs.58992, Hs.591697, Hs.603315, Hs.613351, Hs.642598, Hs.656, Hs.75318, Hs.88523, Hs.89497, Hs.93002, Hs.615092, Hs.62180, Hs.532803, Hs.240, Hs.444028, Hs.58974, Hs.104019, Hs.1594, Hs.178695, Hs.183800, Hs.194698, Hs.20575, Hs.226755, Hs.234545, Hs.239, Hs.244580, Hs.250822, Hs.28465, Hs.368710, Hs.374378, Hs.386189, Hs.436187, Hs.469649, Hs.476306, Hs.482233, Hs.497741, Hs.506652, Hs.509008, Hs.514033, Hs.514527, Hs.592049, Hs.592116, Hs.593658, Hs.631699, Hs.631750, Hs.644048, Hs.72550, Hs.75066, Hs.77695, Hs.83758, Hs.152385, Hs.165607, Hs.203965, Hs.208912, Hs.226390, Hs.26516, Hs.35086, Hs.368563, Hs.403171, Hs.409065, Hs.434886, Hs.436341, Hs.444082, Hs.485640, Hs.498248, Hs.513126, Hs.5199, Hs.520943, Hs.534339, Hs.558393, Hs.567267, Hs.575032, Hs.591046, Hs.591322, Hs.592338, Hs.81892, Hs.83765, Hs.88663, Hs.99480, and Hs.484950;
b. Group B: Genes corresponding to transcripts associated with the Unigene ID Nos. Hs.524571, Hs.226390, Hs.436187, Hs.472716, and Hs.194698;
c. Group C: Genes corresponding to transcripts associated with the Unigene ID Nos. Hs.368563, Hs.444028, Hs.58992, Hs.575032, and Hs.591697;
d. Group D: Genes corresponding to transcripts associated with the Unigene ID Nos. Hs.436187, Hs.194698, Hs.250822, Hs.93002, and Hs.308045;
e. Group E: Genes corresponding to transcripts associated with the Unigene ID Nos. Hs.58992, Hs.522632, Hs.446017, Hs.240, and Hs.533059;
f. Group F: Genes corresponding to transcripts associated with the Unigene ID Nos. Hs.58974, Hs.75318, Hs.506652, Hs.184339, and Hs.81892;
g. Group G: Genes corresponding to transcripts associated with the Unigene ID Nos. Hs.524571, Hs.226390, Hs.436187, Hs.472716, Hs.194698, Hs.386189, Hs.409065, Hs.5199, Hs.434250, and Hs.93002;
h. Group H: Genes corresponding to transcripts associated with the Unigene ID Nos. Hs.368563, Hs.444028, Hs.58992, Hs.575032, Hs.591697, Hs.631750, Hs.250822, Hs.77695, Hs.194698, and Hs.631699;
i. Group I: Genes corresponding to transcripts associated with the Unigene ID Nos. Hs.436187, Hs.194698, Hs.250822, Hs.93002, Hs.308045, Hs.444082, Hs.1594, Hs.184339, Hs.5199, and Hs.409065;
j. Group J: Genes corresponding to transcripts associated with the Unigene ID Nos. Hs.58992, Hs.522632, Hs.446017, Hs.240, Hs.533059, Hs.513126, Hs.132966, Hs.532803, Hs.239, and Hs.58974; and
k. Group K: Genes corresponding to transcripts associated with the Unigene ID Nos. Hs.58974, Hs.75318, Hs.506652, Hs.184339, Hs.81892, Hs.591322, Hs.156346, Hs.72550, Hs.374378, and Hs.77695.

8. The microarray of claim 7, wherein the plurality of nucleic acid probes are capable of detecting the expression of a group of sequences selected from the group consisting of:
l. the sequences SEQ ID NOS: 1-110;
m. the sequences SEQ ID NOS: 27, 85, 62, 23, and 51;
n. the sequences SEQ ID NOS: 88, 45, 31, 102, and 32;
o. the sequences SEQ ID NOS: 62, 51, 57, 40, and 11;
p. the sequences SEQ ID NOS: 31, 26, 22, 44, and 29;
q. the sequences SEQ ID NOS: 46, 37, 67, 6, and 106;
r. the sequences SEQ ID NOS: 27, 85, 62, 23, 51, 61, 90, 97, 18, and 40;
s. the sequences SEQ ID NOS: 88, 45, 31, 102, 32, 75, 57, 79, 51, and 74;
t. the sequences SEQ ID NOS: 62, 51, 57, 40, 11, 93, 48, 6, 97, and 90;
u. the sequences SEQ ID NOS: 31, 26, 22, 44, 29, 96, 3, 43, 55, and 46; and
v. the sequences SEQ ID NOS: 46, 37, 67, 6, 106, 104, 5, 77, 60, and 79.

9. The microarray of claim 8, wherein said plurality of probes each comprise DNA sequences.

10. The microarray of claim 9, wherein said plurality of probes are capable of hybridizing to the sequences of at least one of the groups l-v of claim 5 under the hybridization conditions of 6×SSC at 65° C.

11. The microarray of claim 10, wherein said plurality of probes each comprise from about 15 to 50 base pairs of DNA.

12. A kit comprising the microarray of claim 8 and directions for its use.

13. A method of treating cancer comprising
measuring in a biological sample from a patient the levels of gene expression of a plurality of genes selected from the groups consisting of Group A, B, C, D, E, F, G, H, I, J, and K, defined below:
a. Group A: Genes corresponding to transcripts associated with the Unigene ID Nos. Hs.121025, Hs.126714, Hs.132966, Hs.141125, Hs.156346, Hs.184339, Hs.1973, Hs.270845, Hs.294088, Hs.300701, Hs.308045, Hs.334562, Hs.339665, Hs.369279, Hs.405925, Hs.418533, Hs.433615, Hs.434250, Hs.435570, Hs.436912, Hs.438550, Hs.446017, Hs.472716, Hs.477879, Hs.503749, Hs.522632, Hs.524571, Hs.532968, Hs.533059, Hs.535012, Hs.58992, Hs.591697, Hs.603315, Hs.613351, Hs.642598, Hs.656, Hs.75318, Hs.88523, Hs.89497, Hs.93002, Hs.615092, Hs.62180, Hs.532803, Hs.240, Hs.444028, Hs.58974, Hs.104019, Hs.1594, Hs.178695, Hs.183800, Hs.194698, Hs.20575, Hs.226755, Hs.234545, Hs.239, Hs.244580, Hs.250822, Hs.28465, Hs.368710, Hs.374378, Hs.386189, Hs.436187, Hs.469649, Hs.476306, Hs.482233, Hs.497741, Hs.506652, Hs.509008, Hs.514033, Hs.514527, Hs.592049, Hs.592116, Hs.593658, Hs.631699, Hs.631750, Hs.644048, Hs.72550, Hs.75066, Hs.77695, Hs.83758, Hs.152385, Hs.165607, Hs.203965, Hs.208912, Hs.226390, Hs.26516, Hs.35086, Hs.368563, Hs.403171, Hs.409065, Hs.434886, Hs.436341, Hs.444082, Hs.485640, Hs.498248, Hs.513126, Hs.5199, Hs.520943, Hs.534339, Hs.558393, Hs.567267, Hs.575032, Hs.591046, Hs.591322, Hs.592338, Hs.81892, Hs.83765, Hs.88663, Hs.99480, and Hs.484950;
b. Group B: Genes corresponding to transcripts associated with the Unigene ID Nos. Hs.524571, Hs.226390, Hs.436187, Hs.472716, and Hs.194698;
c. Group C: Genes corresponding to transcripts associated with the Unigene ID Nos. Hs.368563, Hs.444028, Hs.58992, Hs.575032, and Hs.591697;
d. Group D: Genes corresponding to transcripts associated with the Unigene ID Nos. Hs.436187, Hs.194698, Hs.250822, Hs.93002, and Hs.308045;
e. Group E: Genes corresponding to transcripts associated with the Unigene ID Nos. Hs.58992, Hs.522632, Hs.446017, Hs.240, and Hs.533059;
f. Group F: Genes corresponding to transcripts associated with the Unigene ID Nos. Hs.58974, Hs.75318, Hs.506652, Hs.184339, and Hs.81892;
g. Group G: Genes corresponding to transcripts associated with the Unigene ID Nos. Hs.524571, Hs.226390, Hs.436187, Hs.472716, Hs.194698, Hs.386189, Hs.409065, Hs.5199, Hs.434250, and Hs.93002;
h. Group H: Genes corresponding to transcripts associated with the Unigene ID Nos. Hs.368563, Hs.444028, Hs.58992, Hs.575032, Hs.591697, Hs.631750, Hs.250822, Hs.77695, Hs.194698, and Hs.631699;
i. Group I: Genes corresponding to transcripts associated with the Unigene ID Nos. Hs.436187, Hs.194698, Hs.250822, Hs.93002, Hs.308045, Hs.444082, Hs.1594, Hs.184339, Hs.5199, and Hs.409065;
j. Group J: Genes corresponding to transcripts associated with the Unigene ID Nos. Hs.58992, Hs.522632, Hs.446017, Hs.240, Hs.533059, Hs.513126, Hs.132966, Hs.532803, Hs.239, and Hs.58974; and
k. Group K: Genes corresponding to transcripts associated with the Unigene ID Nos. Hs.58974, Hs.75318, Hs.506652, Hs.184339, Hs.81892, Hs.591322, Hs.156346, Hs.72550, Hs.374378, and Hs.77695; and
administering a therapeutically effective amount of one or more cancer treatment agents selected from the group consisting of: cancer chemotherapeutic agents and radiation; or
performing surgery on the patient; or a combination thereof.

14. The method of claim 13, wherein the levels of gene expression are measured by determining the levels of expression of a group of polynucleotide sequences selected from the group consisting of:
l. the sequences SEQ ID NOS: 1-110;
m. the sequences SEQ ID NOS: 27, 85, 62, 23, and 51;
n. the sequences SEQ ID NOS: 88, 45, 31, 102, and 32;
o. the sequences SEQ ID NOS: 62, 51, 57, 40, and 11;
p. the sequences SEQ ID NOS: 31, 26, 22, 44, and 29;
q. the sequences SEQ ID NOS: 46, 37, 67, 6, and 106;
r. the sequences SEQ ID NOS: 27, 85, 62, 23, 51, 61, 90, 97, 18, and 40;
s. the sequences SEQ ID NOS: 88, 45, 31, 102, 32, 75, 57, 79, 51, and 74;
t. the sequences SEQ ID NOS: 62, 51, 57, 40, 11, 93, 48, 6, 97, and 90;
u. the sequences SEQ ID NOS: 31, 26, 22, 44, 29, 96, 3, 43, 55, and 46; and
v. the sequences SEQ ID NOS: 46, 37, 67, 6, 106, 104, 5, 77, 60, and 79.

15. The method of claim 14, wherein the one or more cancer treatment agents are selected from the group consisting of: paclitaxel, docetaxel, imatinib mesylate, sunitinib malate, cisplatin, etoposide, vinblastine, methotrexate, adriamycin, cyclophosphamide, doxorubicin, daunomycin, 5-fluoruracil, vincristine, endostatin, angiostatin, bevacizumab, and rituximab.

16. The method of claim 14, wherein the one or more cancer treatment agents is radiation.

17. The method of claim 14, wherein said cancer is breast, renal, or lung cancer.

18. The method of claim 13, comprising performing surgery on the patient.
